(19) United States
(12) Patent Application Publication
(10) Pub. No.: US 2022/0022765 A1
(43) Pub. Date: Jan. 27, 2022

(54) Heart Condition Treatment and Analysis

(71) Applicant: University of Connecticut, Farmington, CT (US)

(72) Inventors: Ki H. Chon, Mansfield, CT (US); Dong Han, Tolland, CT (US); Syed Khairul Bashar, Mansfield Center, CT (US); Fahimeh Mohagheghian, Storrs, CT (US)

(21) Appl. No.: 17/364,684

(22) Filed: Jun. 30, 2021

Related U.S. Application Data
(60) Provisional application No. 63/046,670, filed on Jun. 30, 2020.
(60) Provisional application No. 63/046,668, filed on Jun. 30, 2020.
(60) Provisional application No. 63/046,664, filed on Jun. 30, 2020.

(51) Int. Cl. A61B 5/024; A61B 5/00; A61B 5/341; G06N 3/02

(52) U.S. Cl. CPC A61B 5/02416; A61B 5/4836; A61B 2562/0219; A61B 5/7264; G06N 3/02; A61B 5/341

(57) ABSTRACT

The present disclosure relates to techniques for the estimating anomalies within photoplethysmographic signals. The techniques may include use of instruments that include photoplethysmographic and acceleration sensors. Time-frequency spectra may be used to determine the anomalies. Corrupted signals may be detected and corrected through applications of machine learning and feature extraction.

### CROSS-REFERENCE TO RELATED APPLICATIONS

This application claims the benefit of U.S. Provisional Application No. 63/046,670, filed Jun. 30, 2020, U.S. Provisional Application No. 63/046,668, filed Jun. 30, 2020, and U.S. Provisional Application No. 63/046,664, filed Jun. 30, 2020, which are incorporated herein by reference in their entirety.

### GOVERNMENT LICENSE RIGHTS

This invention was made with government support under R01HL137734-01 from National Institutes of Health and also under 1522087 from National Science Foundation. The government has certain rights in the invention.

### BACKGROUND

Numerous methods have been devised for the treatment and prevention of atrial fibrillation and other circulatory conditions. In one instance, electrocardiograms are used to analyze electrical impulses to make these determinations. Over treatment and under treatment of these conditions based on electrocardiograms can lead to additional medical complications or unanswered medical needs. Improvements to these determinations may be inefficient when performed on computing devices, preventing real-time or near real-time administration of treatment and analysis.

### SUMMARY

The present disclosure relates to the treatment and analysis of heart and other circulatory conditions. It is to be understood that both the following general description and the following detailed description provide only examples and are not restrictive.

Disclosed is a method. The method may include receiving a photoplethysmographic signal based on a photoplethysmographic sensor. The method may include determining an anomaly of the photoplethysmographic signal relative to a normal sinus rhythm according to a time-frequency representation of the photoplethysmographic signal having a first frequency component. In an example, the first frequency component may have energies within a first range of frequencies.

Also disclosed is an instrument. The instrument may include a photoplethysmographic sensor mounted on the instrument. The instrument may include an accelerometer mounted on the instrument. The instrument may include a processor operable to receive a photoplethysmographic signal based on the photoplethysmographic sensor and an acceleration signal based on the accelerometer and determine an anomaly of the photoplethysmographic signal relative to a normal sinus rhythm, the anomaly determined according to the photoplethysmographic signal and the acceleration signal.

A first example may include a computer-implemented method for estimating heart rate may. The method may include dynamically employing different peak detection methods, as a function of different arrhythmias present in a photoplethysmographic (PPG) signal, to detect peaks in the PPG signal using a combination of the different peak detection methods. The method may include estimating a heart rate based on the peaks detected by the combination. The method may include reporting the heart rate estimated.

The method may further comprise employing an initial peak detection method configured to differentiate a normal sinus rhythm (NSR) from an atrial fibrillation (AF) rhythm to detect an AF rhythm in the PPG signal.

The different peak detection methods may include a peak correction method, the peak correction method including separating fast from normal heart rate AF using a supervised classifier and a threshold value. In an event, the initial peak detection method determines that the AF rhythm is present, the peak correction method may be employed.

In an event, the initial peak detection method determines that the AF rhythm is not present, the method may further include employing a differentiator-adaptive threshold peak detection (DATPD) method.

The different peak detection methods may include a premature atrial contraction (PAC) and premature ventricular contraction (PVC) detection method. The different peak detection may include a heart rate correction method. In an event, the initial peak detection method determines either the NSR or AF rhythm to be present in the PPG signal, the PAC/PVC detection and heart rate correction methods may be employed.

The method may include detecting motion or noise artifacts in a photoplethysmographic (PPG) signal. The method may include separating corrupted PPG data segments from non-corrupted PPG data segments of a plurality of PPG segments of the PPG signal. The separation may be based on the detecting. The corrupted PPG data segments may represent portions of the PPG signal corrupted by the noise or motion artifacts. The features of the non-corrupted PPG data segments may be computed. The non-corrupted PPG data segments may be classified as being associated with an AF rhythm or a normal sinus rhythm as a function of the features computed.

As an example, the detection may include transforming the plurality of PPG segments from a time domain to a frequency domain. The detection may be function of a motion signal and time-frequency spectra (TFS) of the plurality of PPG segments transformed.

A root mean square of successive differences and entropy from beat-to-beat intervals of the non-corrupted PPG segments of the PPG signal may be computed. Classification may include employing a combination of the root mean square of successive differences and entropy computed to distinguish the AF rhythm from the normal sinus rhythm.

The features computed may include time instances, in a frequency domain, of (i) energy of an initial two dominant frequency components of the non-corrupted PPG in a frequency range of the frequency domain and (ii) energy of remaining frequency components in the frequency range.

The method may further comprise identifying premature atrial contraction (PAC) and premature ventricular contraction (PVC) patterns in the non-corrupted PPG segments of the PPG signal and the classifying may be a function of the patterns identified.

### BRIEF DESCRIPTION OF THE DRAWINGS

In order to provide understanding techniques described, the figures provide non-limiting examples in accordance with one or more implementations of the present disclosure, in which:

FIG. 1A illustrates an example instrument for treating a patient with an anomaly from normal sinus rhythm in accordance with one or more implementations of the present disclosure.

FIG. 1B illustrates an example circuitry for treating a patient with an anomaly from normal sinus rhythm is shown in accordance with one or more implementations of the present disclosure.

FIG. 2 illustrates an example method for treating a patient with an anomaly from normal sinus rhythm in accordance with one or more implementations of the present disclosure.

FIG. 3 illustrates a waveform envelop peak detection method in accordance with one or more implementations of the present disclosure.

FIG. 4 illustrates an expanded step that may be used for selecting or inverting the top or bottom portion of the window of the PPG signal in accordance with one or more implementations of the present disclosure.

FIG. 5A illustrates a PPG signal 126 and an ECG signal 502 over time in accordance with one or more implementations of the present disclosure.

FIG. 5B, illustrates various signals in accordance with one or implementations of the present disclosure.

FIG. 6 illustrates a combination of plots in accordance with one or more implementations of the present disclosure.

FIG. 7 illustrates a scatter plot 700 of mean distances relative the ratio of points inside the region 608 in accordance with one or more implementations of the present disclosure.

FIG. 8 illustrates an example correction for atrial fibrillation with (rapid ventricular response) RVR or fast heart rate atrial fibrillation (FAF) in accordance with one or more implementations of the present disclosure.

FIG. 9 illustrates an example of PVC and PAC detection in accordance with one or more implementations of the present disclosure.

FIG. 10 illustrates a sequence of frequency components from a bandpass filtered PPG signal 126 in the frequency domain in accordance with one or more implementations of the present disclosure.

FIG. 11 illustrates an example of the peak correction in accordance with one or more implementations of the present disclosure

FIG. 12A illustrates a clean PPG signal and a corrupted PPG signal in accordance with one or more implementations of the present disclosure.

FIG. 12B illustrates a corrupted PPG signal in accordance with one or more implementations of the present disclosure.

FIG. 13A illustrates a corrupted PPG signal based on acceleration in accordance with one or more implementations of the present disclosure.

FIG. 13B illustrates an acceleration signal in accordance with one or more implementations of the present disclosure.

FIG. 14A illustrates a PPG signal in accordance with one or more implementations of the present disclosure.

FIG. 14B illustrates a time frequency spectrum with respect to the PPG signal in accordance with one or more implementations of the present disclosure.

FIG. 15A illustrates a noisy PPG signal in accordance with one or more implementations of the present disclosure.

FIG. 15B illustrates a noisy time-frequency spectrum in accordance with one or more implementations of the present disclosure.

FIG. 16A illustrates the quantified energy within the primary frequency components in in accordance with one or more implementations of the present disclosure.

FIG. 16B illustrates the quantified energy within the secondary frequency components in in accordance with one or more implementations of the present disclosure.

FIG. 16C illustrates the quantified energy within the remaining frequency components in in accordance with one or more implementations of the present disclosure.

FIG. 17A illustrates a PPG signal 126 over a window 128 that is detected as clean in accordance with one or more implementations of the present disclosure.

FIG. 17B illustrates a PPG segment 126 over a window 128 that is detected as corrupted in accordance with one or more implementations of the present disclosure.

FIG. 18A illustrates a PPG signal 126 over a window 128 that is detected as clean with atrial fibrillation in accordance with one or more implementations of the present disclosure.

FIG. 18B illustrates a PPG segment 126 over a window 128 that is detected as corrupted in accordance with one or more implementations of the present disclosure.

FIG. 19A illustrates a substantially constant heart rate over time that depicts normal sinus rhythm in accordance with one or more implementations of the present disclosure.

FIG. 19B illustrates an irregularly irregular heart rate over time that depicts atrial fibrillation in accordance with one or more implementations of the present disclosure

FIG. 20A illustrates a plot is shown indicating relative placements between windows of SampEn with respect to RMSSD in accordance with one or more implementations of the present disclosure.

FIG. 20B illustrates an indication of the respective atrial fibrillation determination is also presented in accordance with one or more implementations of the present disclosure.

FIG. 21 illustrates an example method for determining atrial fibrillation in accordance with one or more implementations of the present disclosure.

FIG. 22 illustrates a reduction in unnecessary treatments and missed treatments in accordance with one or more implementations of the present disclosure.

FIG. 23 illustrates a method for PVC and PAC determination in accordance with one or more implementations of the present disclosure.

FIG. 24A illustrates a normal sinus rhythm panel in accordance with one or more implementations of the present disclosure.

FIG. 24B illustrates a PVC panel in accordance with one or more implementations of the present disclosure.

FIG. 25A illustrates a normal heart rate atrial fibrillation (NAF) panel, or basal heart rate atrial fibrillation, over a window 128 (e.g., 30 seconds) in accordance with one or more implementations of the present disclosure.

FIG. 25B illustrates a fast heart rate atrial fibrillation (FAF) panel, or atrial fibrillation with rapid ventricular response, over a window 128 (e.g., 30 seconds) in accordance with one or more implementations of the present disclosure.

FIG. 26A illustrates a 1-2-3 pattern panel over a window 128 (e.g., 30 seconds) in accordance with one or more implementations of the present disclosure.

FIG. 26B illustrates a 6-4-5 pattern for fast heart rate PAC panel over a window 128 (e.g., 30 seconds) in accordance with one or more implementations of the present disclosure.

FIG. 26C illustrates a 6-4-5 pattern for fast heart rate PVC panel over a window 128 (e.g., 30 seconds) in accordance with one or more implementations of the present disclosure.

FIG. 27A illustrates a 2-4 pattern panel for fast heart rate PAC with trigeminy over a window 128 (e.g., 30 seconds) in accordance with one or more implementations of the present disclosure.

FIG. 27B illustrates a 2-4 pattern panel 2630 for fast heart rate PAC with bigeminy over a window 128 (e.g., 30 seconds) in accordance with one or more implementations of the present disclosure.

FIG. 28 illustrates pseudocode for PAC and PVC detection of atrial fibrillation windows is shown in accordance with one or more implementations of the present disclosure

FIG. 29 illustrates a first portion of pseudocode for PAC and PVC detection on non-atrial fibrilliation windows is shown in accordance with one or more implementations of the present disclosure

FIG. 30 illustrates a second portion of pseudocode for PAC and PVC detection on non-atrial fibrilliation windows is shown in accordance with one or more implementations of the present disclosure.

FIG. 31A illustrates classification of PAC and PVC according to features of the underlying PPG signal in accordance with one or more implementations of the present disclosure.

FIG. 31B illustrates different PAC and PVC beats and demonstrates the features of the underlying PPG signal in accordance with one or more implementations of the present disclosure.

FIG. 32 illustrates three amplitude and time interval measures based on consecutive PPG signal peaks in accordance with one or more implementations of the present disclosure.

FIG. 33 illustrates a selection method in accordance with one or more implementations of the present disclosure.

FIG. 34 illustrates an autoencoder or a network of encoders and decoders in accordance with one or more implementations of the present disclosure

FIG. 35 illustrates a denoised PPG signal after reconstruction by the autoencoder in accordance with one or more implementations of the present disclosure.

FIG. 36 illustrates a training method 3600 in accordance with one or more implementations of the present disclosure.

FIG. 37 illustrates a trained model to provide the denoised PPG signal 3506 in accordance with one or more implementations of the present disclosure.

### DETAILED DESCRIPTION

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a special purpose computer or other programmable data processing instrument to produce a machine, such that the processor-executable instructions which execute on the computer or other programmable data processing instrument create a device for implementing the functions specified in the flowchart block or blocks.

These processor-executable instructions may also be stored in a computer-readable memory or a computer-readable medium that may direct a computer or other programmable data processing instrument to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing instrument to cause a series of operational steps to be performed on the computer or other programmable instrument to produce a computer-implemented process such that the processor-executable instructions that execute on the computer or other programmable instrument provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for using a machine learning classifier(s) for detection and classification of objects. Machine learning (ML) is a subfield of computer science that gives computers the ability to learn through training without being explicitly programmed. Machine learning methods include, but are not limited to, deep-learning techniques, naïve Bayes classifiers, support vector machines, decision trees, neural networks, and the like.

The method steps recited throughout this disclosure may be combined, omitted, rearranged, or otherwise reorganized with any of the figures presented herein and are not intend to be limited to the four corners of each sheet presented.

Photoplethysmographic (PPG) sensors may be used to convey information from smart watches and other devices to determine heartrate, blood oxygenation, blood sugar, and other information. Light-emitting diodes can be mounted on smart watches and other devices to generate PPG signals and data. As discussed throughout this disclosure, PPG signals can be used to detect abnormalities, or an anomaly from a normal sinus rhythm, in the PPG signal for indication and treatment by medical professionals. As an example, atrial fibrillation is an irregular and often rapid heart rate that occurs when the two upper heart chambers experience chaotic electrical signals, which commonly causes poor blood flow. Consequences associated with untreated atrial fibrillation can be dire. For instance, blood clots, stroke, and heart failure are all complications of atrial fibrillation. Overtreatment of atrial fibrillation due to false positives can also lead to additional complications. In such a way, it is important to reduce predictive errors associated with PPG sensors to ensure proper treatment is administered.

Additionally, improvements to the overtreatment and undertreatment of these conditions may be inefficient, especially when used in conjunction with wearables and other devices that have processing and power constraints. As such, an improvement to the efficiency or operation of these systems that improves accuracy and reduces the overtreatment and undertreatment of these conditions is disclosed.

A patient with atrial fibrillation basal heart rate can be either within a normal normal range or fast. The detection of a heart rate with a normal sinus rhythm may noticeably different from a heart rate with atrial fibrillation leading to additional false positives and negatives. Poor signal-to-noise ratios of the PPG signal may further compound detection of heart rates and atrial fibrillation. As an example, skin complexion and blood perfusion within arteries can affect the quality of signals from the PPG sensor. Motion of the PPG sensor can further impact the determination of atrial fibrillation, especially when disguised as premature ventricular contractions (PVC) or premature atrial contractions (PAC). It is clinically important to distinguish between these different anomalies as a diverse set treatments may be prescribed and administered for atrial fibrillation subjects who present with fast heart rates but not for those with PVC or PAC. This diverse set of treatments may be administered based on confidence in the atrial fibrillation determination.

An example treatment may include electrical cardioversion where an electrical shock is delivered to the heart. As another, an anti-arrhythmic medication may be administered or prescribed. These treatments may cause side effects. As another treatment, a beta blocker, calcium channel blocker or digoxin may be administered. Notwithstanding the administration of catheter and surgical procedures that may include ablation and other treatments. As such, the accuracy of detection along with the proper treatment is paramount. This broad range of treatments, and others not mentioned, including anticoagulants, are contemplated throughout this disclosure, providing clinicians with a diverse selection of options to ward off disease. These options may be integrated and combined to improve patient outcomes.

The techniques disclosed herein improve the treatment of patients with a variety of conditions including but not limited to atrial fibrillation, rapid ventricle rates, premature atrial contraction, premature ventricle contraction, and other conditions that depart from normal sinus rhythms. As discussed herein, these conditions may or may not be specific but rather general categories of conditions that may require treatment. Additionally, a normal sinus rhythm may not be specific but rather a general category of conditions that do not require treatment.

The techniques disclosed herein may be implemented on a computing device in a way that improves the efficiency of its operation. As an example, the methods, instructions, and steps disclosed herein may improve the functioning of a computing device and also reduce error rates associated with detecting these conditions accurately. These reduced errors can improve the resulting administration of treatment by practitioners or by instruments.

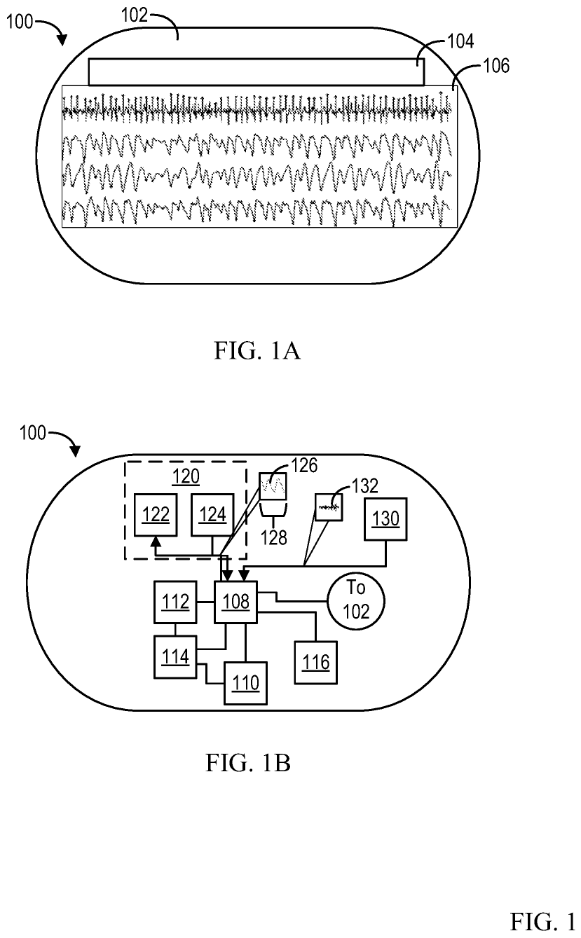

Referring to FIG. 1A, an example instrument 100, also referred to herein as an apparatus, for treating a patient with an anomaly from normal sinus rhythm is shown in accordance with one or more implementations of the present disclosure. The instrument 100 may be a standalone device or integrated with other components. As an example, the instrument 100 may be integrated within a watch or another wearable. The instrument 100 may be integrated or position within a bed or room of a patient. As another non-limiting example, the instrument may be prescribed along with medications to indicate times for administration or automatic emergency assistance.

The instrument 100 may include a display 102 for providing information to patients and staff. As an example, the display 102 may include an indication bar 104 to provide indications related to raw sensory data, determined patient conditions, instrument status, or combinations thereof. The display 102 may include a graphical portion 106 configured to output electrocardiogram or PPG signals. The signals may be outputted in real-time or pseudo real-time.

In FIG. 1B, an example circuitry for treating a patient with an anomaly from normal sinus rhythm is shown in accordance with one or more implementations of the present disclosure. The instrument 100 may include circuitry to detect, treat, or combinations thereof anomalies with respect to a normal sinus rhythm of a patient. As an example, the circuitry may include one or more processors 108 referred to herein as processor 108. The processor 108 may be operable to execute instructions stored on a computer-readable medium 110. The instructions may be defined through software or hardware, as stored machine code, assembly instructions, application specific integrated circuits, programmable logic or gates, other circuitries or instructions, and combinations thereof. The computer-readable medium may include non-transitory information. The computer-readable medium 110 may be various implements and combinations, including but not limited to, persistent and non-persistent memory, burned-in and removable memory, and magnetic and electronic memory. The processor 108 may be configured for off-board communication according to network circuitry 112. The network circuitry 112 may interface with other instruments, treatment apparatuses, data storage, displays, and other components associated with patient diagnosis and treatment. As an example, the network circuitry 112 may include a BLUETOOTH interface, 802.3 interface, 802.11 interface, nearfield interface, antennae, other communication circuitry, and combinations thereof to provide integration with other instruments, treatment apparatuses, data storage, displays, and other components associated with patient diagnosis and treatment.

The instrument 100 may be powered internally or externally. As an example, the instrument 100 may be powered by an internal battery 114. The internal battery 114 may be directly or indirectly connected to power the network circuitry 112, the processor 108, the memory 110, and other components associated with the instrument 100. The processor 108 may be configured to execute treatment or treatment plans based on PPG signals. As an example, the processor 108 may be associated with defibrillation circuitry 116 or other circuitry (e.g., pacemaker) configured to the treat a patient on command.

The processor 108 may be configured to communicate with or operate a PPG sensor 120. Various sensory configurations are contemplated by this disclosure. As an example, the processor 108 may be configured to send electrical signals to a light emitting diode (LED) 122. The LED 122 may emit photons or light energy to the patient. A light sensor 124 may receive the reflected photons or light energy and generate an electrical PPG signal 126 received by the processor 108. In an example, the processor 108 is operable to execute instructions to detect anomalies within the PPG signal 126 to operate the defibrillation circuitry 116 or other circuitry to treat the patient according to the received PPG signal 126. The PPG signal may be stored as a window 128. The window 128 may be 30 seconds. Other window sizes are contemplated by this disclosure. The window or segment may be sliding, that is, the oldest segment of the window is dropped according to time increment and the newest segment of the PPG signal 126 is added according to time increment. As an example, a 30-second window may contain data broken into various increments or sampling rates (e.g., 10 ms). The last 10 ms of data is dropped every time a new 10 ms of data is received. The processor may communicate with the PPG sensor 120 through various protocols including electrical signaling and digital communications.

The processor 108 may be configured to communicate with or operate an accelerometer 130 or other sensors. In an example, the accelerometer 130 provides an acceleration signal 132 to the processor 108. The acceleration signal 132 may be communicated with electrical signaling, digital communications, or combinations thereof. The acceleration signal 132 may indicate acceleration in more than one dimension. As one example, the acceleration signal 132 indicates three-dimensional components (e.g., x, y, z) of acceleration associated with the instrument 100 or the patient.

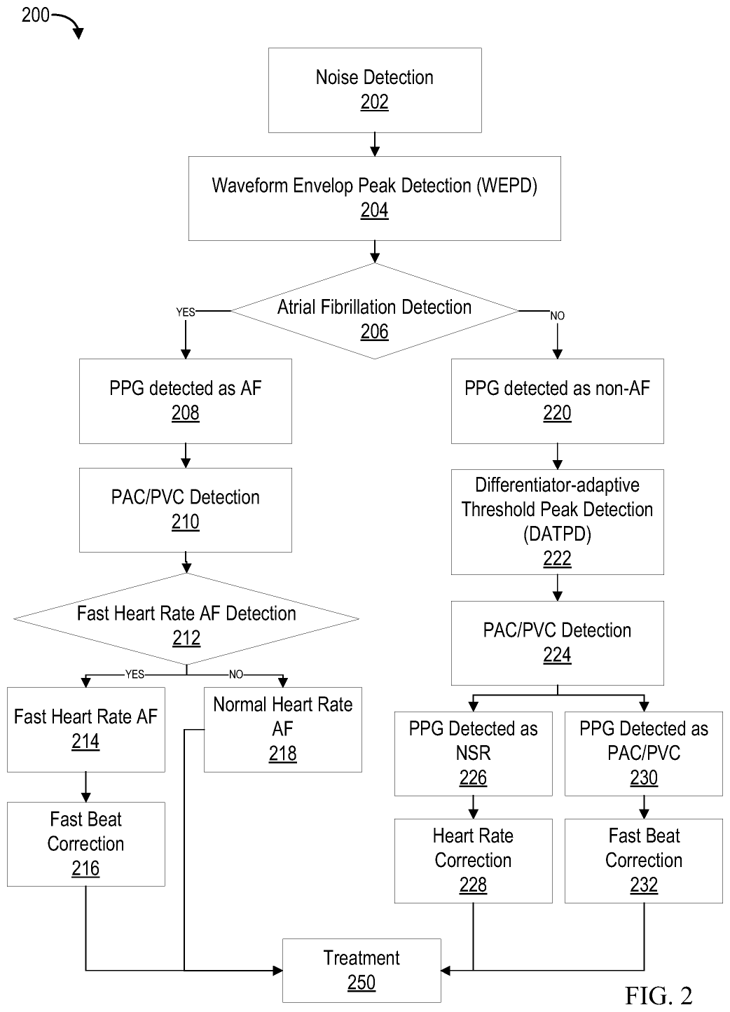

Referring to FIG. 2, an example method 200 for treating a patient with an anomaly from normal sinus rhythm is shown in accordance with one or more implementations of the present disclosure. Although the order or execution of steps may be rearranged, the method 200 may include selecting clean PPG signals for analysis, detecting peaks to reduced false negative atrial fibrillation detection, and false positive detection of normal sinus rhythms. The method 200 may include PAC and PVC detection and further determination of atrial fibrillation according to the heart rate. The steps disclosed herein may be distinctly or generally defined with combinations of other steps and operations or portions thereof. As an example, selection of clean PPG signals may comprise a quantity of steps and waveform envelop peak detection may similarly comprise a quantity of steps. Although designated as part of the selection step or the peak detection step, these intermediate steps may be performed as part of the more generalized selection, peak detection steps, or combinations of the selection and peak detection steps.

In step 202, the noise detection may be performed to detect motion artifacts, noise, or other combinations within the PPG signal 126. Noise detection may include selection of clean segments of the PPG signal 126. As an example, noisy sections may be detected and cleaned, ignored, or discarded, as discussed herein.

In step 204, a first peak detection may be performed. As an example, the first detection of peaks may define the beats within the PPG signal 126. The first detection of peaks may be implemented with a waveform envelop peak detection (WEPD) method. Other methods are also contemplated.

In step 206, a first detection of atrial fibrillation may be determined. In one aspect, atrial fibrillation may be defined as an irregularly irregular heart rate. In other words, atrial fibrillation may be defined as a random sequence of heart beat intervals with increased beat-to-beat variability and complexity. Atrial fibrillation may be determined based on a sequence of data points taken from PPG signal 126. As an example, the data points may be defined over a thirty-second interval. As the methods described herein look for randomness and other indicators of atrial fibrillation, it should be appreciated that accurate peak intervals may be unnecessary.

In step 208, the PPG signal 126 is determined as corresponding to atrial fibrillation. In step 210, PAC and PVC determinations are performed on the PPG signal 126. The PAC and PVC detection in step 210 may be different for atrial fibrillation than compared with a signal detected to have a normal sinus rhythm. If PAC or PVC is detected the method may end. In step 212, a detection of the heart rate speed is detected. The detection may include detection of a rapid ventricle rate (RVR), or fast heart rate atrial fibrillation (FAF). In order to detect fast heart rates with atrial fibrillation, a trained classifier may be used. In some instances, the classifier is trained with a supervised process.

Based on the determination in step 212, a fast heart rate with atrial fibrillation may be determined in step 214 or a normal heart rate with atrial fibrillation in step 218. In step 216, the fast heart rate with atrial fibrillation is corrected to compensate for the fast heart rate detected in step 212. As an example, the local minima of interspersed heart rates may be missed through the preceding detection methods, fast heart beat correction in step 216 may serve to correct the heart rate errors to provide a proper indication of atrial fibrillation and an accurate heart rate. The corresponding information may be used in step 250 for treatment of the underlying disease as discussed herein.

Continuing with the non-atrial fibrillation PPG signal 126 in step 220, heart rate patterns may be detected using a differentiator-adaptive threshold peak detection algorithm. In such an instance, PAC or PVC detection is performed on the PPG signal 126 having non-atrial fibrillation characteristics. If PAC or PVC is detected as shown step 230, fast beat correction may be performed to add missed beats from the previous steps within the PPG signal 126. If a normal sinus rhythm is detected as shown in step 226, a heart rate correction may be performed to ensure that motion artifacts or noise artifacts are not providing incorrect heart rate determinations in step 228.

The treatment in step 250 may include performing operations based on the corrected heart rates and abnormal conditions detected. The method 200 may provide an indication of atrial fibrillation, with or without RVR, PAC, PVC, normal sinus rhythm, along with the detected heart rate. These factors may be used to determine and administer the proper treatment for the patient.

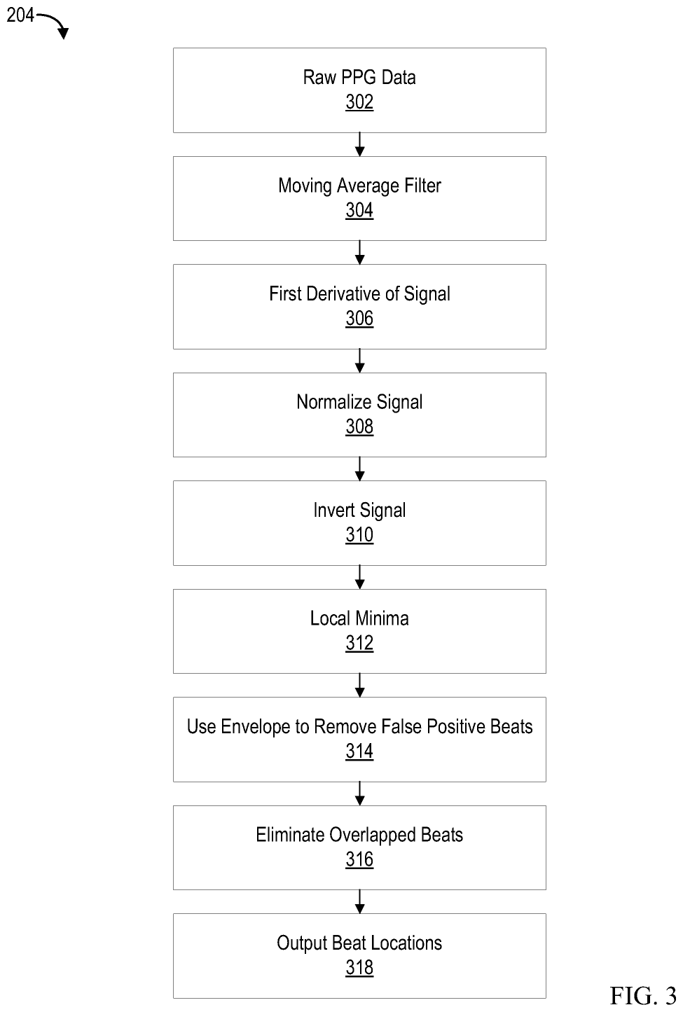

Referring to FIG. 3, a waveform envelop peak detection method 204 is shown in accordance with one or more implementations of the present disclosure. The waveform envelop peak detection method may be referred to as a peak detection method 204 throughout this disclosure and as indicated in step 204 of FIG. 1B. In step 302 raw PPG data is received. The raw PPG data may be the PPG signal 126. In another example, the raw PPG data is based on the PPG signal 126. The PPG signal 126 may be digital or analog, and may be digitized before it is received in step 302. For brevity, the data that is based on the PPG signal 126 or the PPG signal 126 itself will be referred to as the PPG signal 126. The PPG signal 126 may be clean or determined to be free from motion or noise artifacts. As an example, other methods and steps discussed herein may be used to determine that the PPG data is clean or determined to be free from motion or noise artifacts.

In step 304, a moving average filter is applied to the PPG data. In this step or another, a bandpass filter may be applied with a passband. The passband may be 0.5 Hz to 5 Hz. As one example, the bandpass filter may be based on a fifth order zero phase elliptic filter. The bandpass filter may be applied to the window 128 of the PPG data. For example, the bandpass filter may be applied the PPG data according to the window 128.

The moving average filter may ensure the smoothness of the signal within the window 128. The moving average filter may be used to remove non-cardiac oscillations and spikes caused by motion artifacts or poor signal quality. The moving average filter may be implemented an equation (e.g., Equation 1).

$$b ( i - M + 1 ) = \frac{1}{2 M + 1} {\sum }_{j = 1 - m}^{i + M} a ( j ) \qquad ( 1 )$$

where i=M, M+1, M+2 . . . N−M−1 with M=fs/10 for the first moving average process, and M=fs/9 for the remaining two times; a(j)=1, 2, 3 . . . N denotes the raw signal after bandpass filtering; and fs is the sampling rate.

In step 306, in between one of the smoothing iterations (e.g., before the final smoothing iteration), calculation of the first derivative of the filtered signal may performed to accentuate small fluctuations in the peak plateau portion of the PPG waveform in Equation 2:

c(i)=b(i+1)−b(i) (2)

In step 308, the window 128 may be normalized. The mean of c(i) is subtracted from c(i) and divided by the standard deviation of c(i). As indicated in Equation 3.

$$d ( i ) = \frac{c ( i ) - mean ( c )}{s t d ( c )} \qquad ( 3 )$$

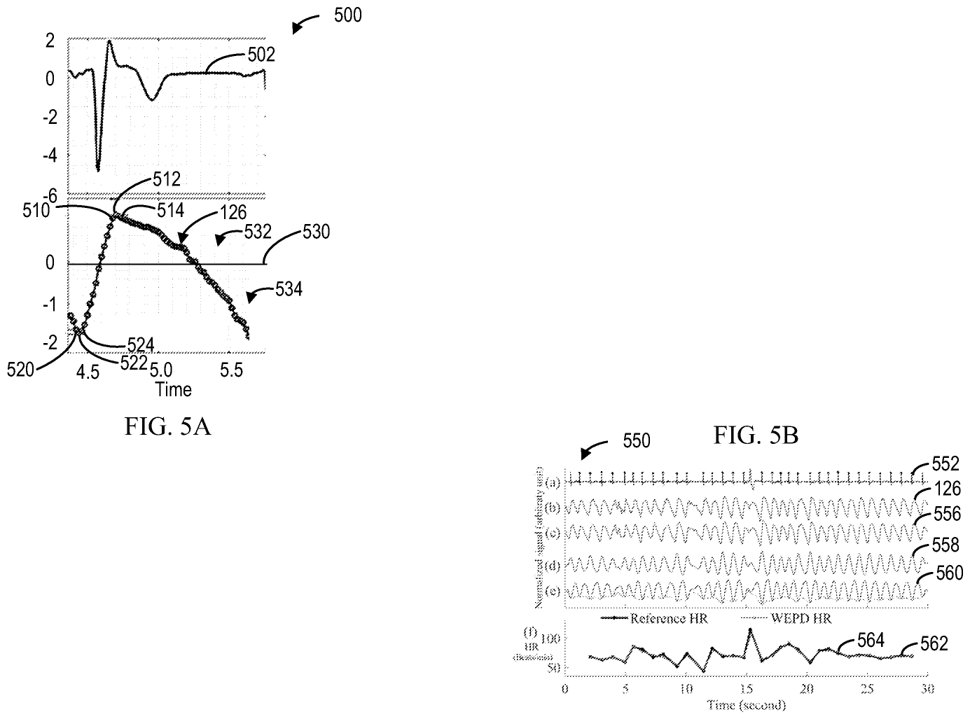

Step 308 may enable a waveform envelope calculation to remove false positive beats as described in this disclosure. Peak detection may be applied to the top portion 532 or bottom portion 534 of the window 128 of the PPG signal 126, as indicated in FIG. 5A. FIG. 5A illustrates a PPG signal 126 and an ECG signal 502 over time, in accordance with one or more implementations of the present disclosure. For example, peak detection may be applied to a curve portion that has the sharpest peaks to increase or maximize accuracy of the heart rate estimation. Various methods may be used to identify the sharpest peaks. As one example, a minimum height of the peaks may be set as the mean of the window 128.

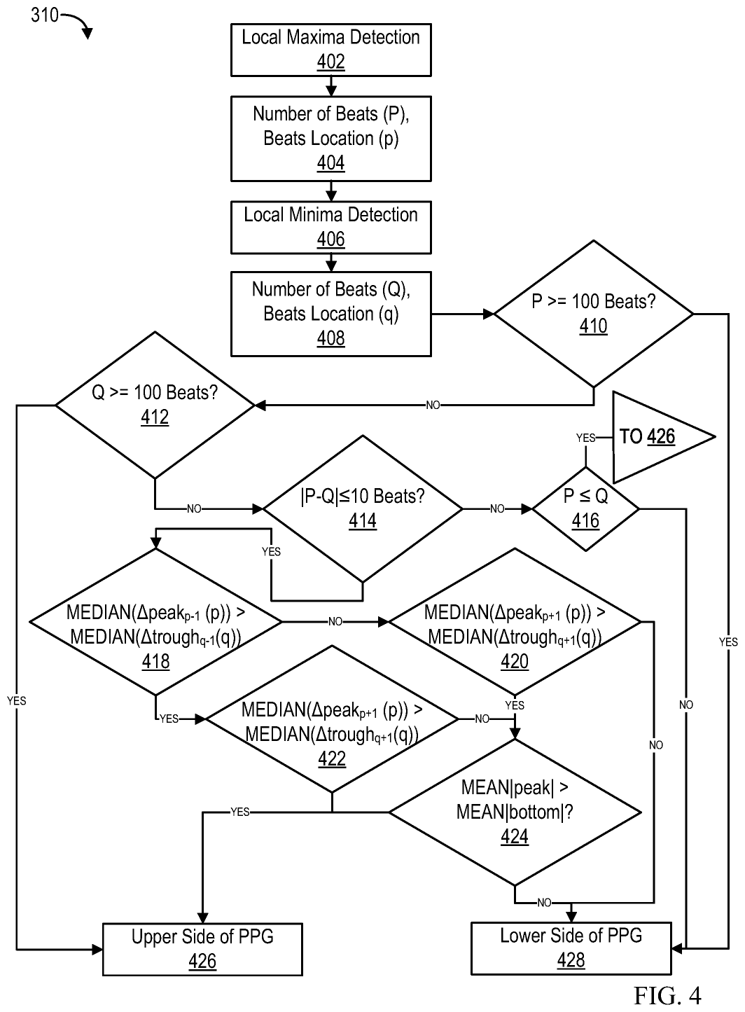

In step 310 the window 128 may be screened by determining whether there are more than a quantity of peaks (e.g., 100) within the top portion 532 of the signal or the bottom portion 534 of the PPG signal 126. The top portion 532 may be defined above the mean 530 of the PPG signal 126 within the window, and the bottom portion may be defined below the average value of the PPG signal 126 within the window. If there are more than the quantity of peaks within the top portion of the signal or the bottom portion, the side with less than 100 peaks is selected. As one example, if the quantity of peaks of the top is disparate from the quantity of peaks on the bottom (e.g., greater than 10 peaks) then the side, top or bottom, of the window 128 with the fewest peaks is used. As an example, FIG. 4 provides an expanded step 310 that may be used for selecting or inverting the top or bottom portion of the window 128 of the PPG signal 126 in accordance with one or more implementations of the present disclosure. In step 402, local maxima 512 is detected as shown in FIG. 5A, local maxima 512 of the PPG signal 126. The local maxima 512 may be determined based on the mean 530 of the window 128. Data within a segment (e.g., between 4.5 and 5.5 seconds) above the mean 530 may be associated with a local maxima 512, while data within the segment below the mean 530 may be associated with local minima 522. As such, the segment may be determined between where the PPG signal 126 intersects the mean 530.

In step 404, the quantity of beats, P, of the window 128 is determined along with the local maxima 512, p, of the segment, as shown in FIG. 5A. In step 406, the local minima 522 is detected as the minimum value associated with the segment. In step 408, the quantity of beats, Q, of the window 128 is determined along with the local maxima 512, q, of the segment, as shown in FIG. 5A. Otherwise, further determination may be used to select either the top portion 532 or the bottom portion 534.

In step 410, the processor 108 or another implement can determine whether the quantity of beats, P, associated with the local maxima 512 is greater than a quantity of peaks beyond a typical heart rate range. As an example, if a typical heart rate does not exceed 200 beats per minute, then the beats in a 30-second window should not exceed 100. The quantity of peaks may be set to 100. A similar quantity, but not necessarily the same, may be used with regard to the quantity of beats, Q, associated with the local minima 522. If the quantity of peaks, P or Q, is greater than the set quantity, the other side of the PPG signal 126 within the window 128 is used. For instance, if the quantity of beats, Q, associated with the local minima 522 is greater than the set quantity based on the typical heart rate range, the top portion 532 or upper side of the PPG signal 126 is used. Continuing with step 414 if P and Q are less than their respective set quantities, a determination of the absolute value of the difference between P and Q is determined and compared with a deadband quantity configured to determine whether P and Q are quantifiably different. If the absolute value of the difference is greater than the deadband quantity the top portion 532 or bottom portion 534 is selected by the lesser of the quantities P and Q in step 416. If P and Q are substantially similar, four types of amplitude gradients are calculated: |Δpeakₚ₋₁(p)|, |Δpeakₚ₊₁(p)|, |Δtrough_{q+1}(q)|, and |Δtrough_{q-1}(q)|. These amplitude gradients are based on the absolute amplitude changes between one sample before 510, 520 and one sample after 514, 524 the local maxima and local minima positions, respectively, where p=1, 2 . . . P and q=1, 2 . . . Q. The median of these indices is also calculated. In such a way, the sharper of the peaks may be calculated. As an example, in step 418 the median of |Δpeakₚ₋₁(p)| is compared to the median of Δtrough_{q-1}(q).

If the median of |Δpeakₚ₋₁(p)| is less than the median of Δtrough_{q-1}(q), the median of |Δpeakₚ₊₁(p)| is compared with the median of |Δtrough_{q+1}(q)| in step 420. If the median of |Δpeakₚ₊₁(p)| is greater than the median of |Δtrough_{q+1}(q)|, a mean analysis is used to determine the sharper of the peaks in step 424. Otherwise, the lower side of the PPG signal 126 is used.

If the median of |Δpeakₚ₋₁(p)| is greater than the median of Δtrough_{q-1}(q), the median of |Δpeakₚ₊₁(p)| is compared with the median of |Δtrough_{q+1}(q)| in step 422. If the median of |Δpeakₚ₊₁(p)| is less than the median of |Δtrough_{q+1}(q)|, a mean analysis is used to determine the sharper of the peaks in step 424. Otherwise, the upper side of the PPG signal 126 is used.

In step 424, the median of the gradient values is not determinative. As such, the mean may be used as a tiebreaker. As an example, if the mean of the peak values is greater than the mean of the trough values the top portion of the PPG signal 126 is used, as shown in step 426. Otherwise, the bottom portion of the PPG signal 126 is used, as shown in step 428. The mean of the peak values and the mean of the trough values may be calculated according to Equations 4 and 5, respectively.

mean|peak|=median|Δpeakₚ₋₁(p)|+median|Δpeakₚ₊₁(p)| (4)

mean|trough|=median|Δtroughₚ₋₁(p)|+median|Δtroughₚ₊₁(p)| (5)

As such, referring back to step 310, if the top portion 532 of the PPG signal 126 is selected, the PPG signal 126 is inverted over the mean 530 and then the local minima 522 is estimated for the beats within the inverted top portion 532. As an example, FIG. 5B, provides various signals in accordance with one or implementations of the present disclosure. As an example, a raw electrocardiogram (ECG) signal 552 with reference ECG beats is shown. A raw PPG signal 126 is shown have substantially similar peaks to that of the ECG signal 552. After the bandpass filter, the bandpass filtered PPG signal 556 is shown. In addition, the output after moving average filtering in step 304 is shown as moving average PPG signal 558. If the top portion 532 is selected, the inverted, normalized signal 560 is shown. As shown, the inverted, normalized signal 560 has opposite peaks as the moving average PPG signal 558. The method 204 may identify the estimated heart rate 564, which is shown respective to the ECG heart rate 562 with minimal appreciable difference.

Step 314 of FIG. 3 removes false positive beats using an envelope. A dicrotic notch may be prevalent in PPG signals 126 having a normal sinus rhythm. Motion artifacts can also crate the appearance of a dicrotic notch. If these dicrotic notches are not accounted for, they may be detected as a heart rate and can also lead to the false detection of atrial fibrillation. As an example, an envelope of the PPG signal 126 may be estimated. A second local minima that corresponds to the dicrotic notch is identified where the local minima that intersects the envelope is the new estimated peak (e.g., local maxima 512) for the segment.

In step 316, overlapped peaks of the PPG signal 126 may be eliminated. As an example similar to the removal of false positive beats in step 314, only the lowest amplitude peak, local maxima 512 is retained. As an example, a segment may have more than one peak. A calculation of local maxima 512 is calculated for each peak and the lowest peak is retained as the peak for that segment. A period (e.g., 0.3 seconds) may be selected to ensure that consecutive heartbeats are not too closely spaced. The period may be based on the refractory period of cardiac muscle for normal sinus rhythms. The resulting beats may be output to identify the estimated heart rate 564 in step 318.

Back to step 212, of FIG. 2, a detection of the heart rate speed is detected. The detection may include detection of a rapid ventricle rate (RVR). As classifier may be used to determine atrial fibrillation with a rapid ventricle response. The classifier may be trained with supervision. Atrial fibrillation with RVR may be indicated by a heart rate that is greater than 140 BPM and a heart rate that increases more than 40 BPM from one beat to the next. Clinically, a determination for atrial fibrillation with RVR may be indicated by and a heart rate output from step 204 that failed to follow more than half of the reference heart rate 562.

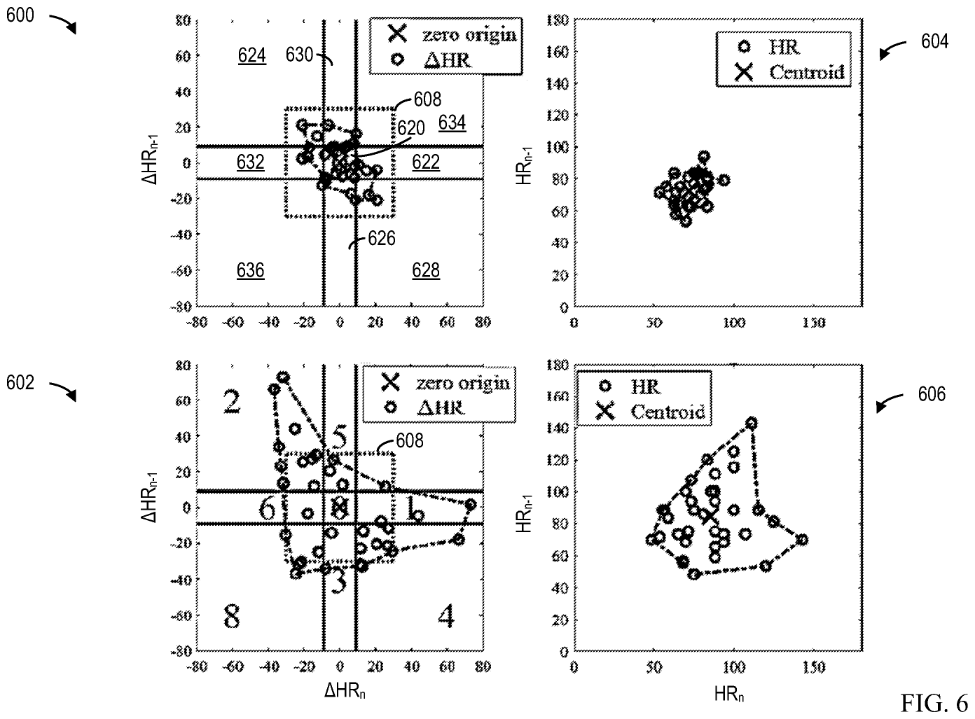

In FIG. 6, a combination of plots are shown in accordance with one or more implementations of the present disclosure. The plots may be considered of the Poincare type, indicating the heart rate difference between a basal atrial fibrillation in plot 600 and atrial fibrillation with RVR in plot 602. The Poincare plot further indicates the heart rate associated with basal atrial fibrillation in plot 604 and atrial fibrillation with RVR in plot 606. Various features of the PPG signal 126 may be used to indicate atrial fibrillation with RVR instead of basal atrial fibrillation. A count of the quantity of beats within the window 128 that exceed 140 beats per minute (BPM) may indicate atrial fibrillation with RVR. Plots 600, 602 may further indicate five other features, plots 604, 606 may indicate three features. Four of the five features derived from plots 600, 602 may be based on a calculation of distance between each point to the origin, providing the mean value of all distances, the variance of all distance, a boundary of the covered area of all points, and a ratio of points that have a distance to other points that is less than 5 BPM. Lastly, a feature may be the ratio of points that fall inside the RVR region 608 and outside of the RVR region 608. In another example, the feature may be the ratio of points that fall inside the RVR region 608 and the total quantity of points of the PPG signal 126. The RVR region 608 may be defined from negative 30 to positive 30 difference in heart rate for three consecutive heartbeats.

The plots 604, 606 may have an X-axis that represents the n+1 heart rate and the Y-axis representing the current heart rate. As another example, the Y-axis may represent the n−1 heart rate and the X-axis may represent the current heart rate. The centroid may be calculated by averaging the coordinates of all points plotted. In one example, the mean value of all distances of points plotted may be used separate the basal atrial fibrillation from the atrial fibrillation with RVR. The plots 600, 602 may have an X-axis that represents the n+1 heart rate difference between three consecutive heartbeats and the Y-axis representing the current heart rate difference between three consecutive heartbeats. As another example, the Y-axis may represent the n−1 heart rate difference between three consecutive heartbeats and the X-axis may represent the current heart rate difference between three consecutive heartbeats. The centroid may be calculated by averaging the coordinates of all points plotted. Although shown graphically for demonstration, the processor 108 may calculate the features without plotting, determining the feature values directly from the PPG signal 126 or derived PPG signals.

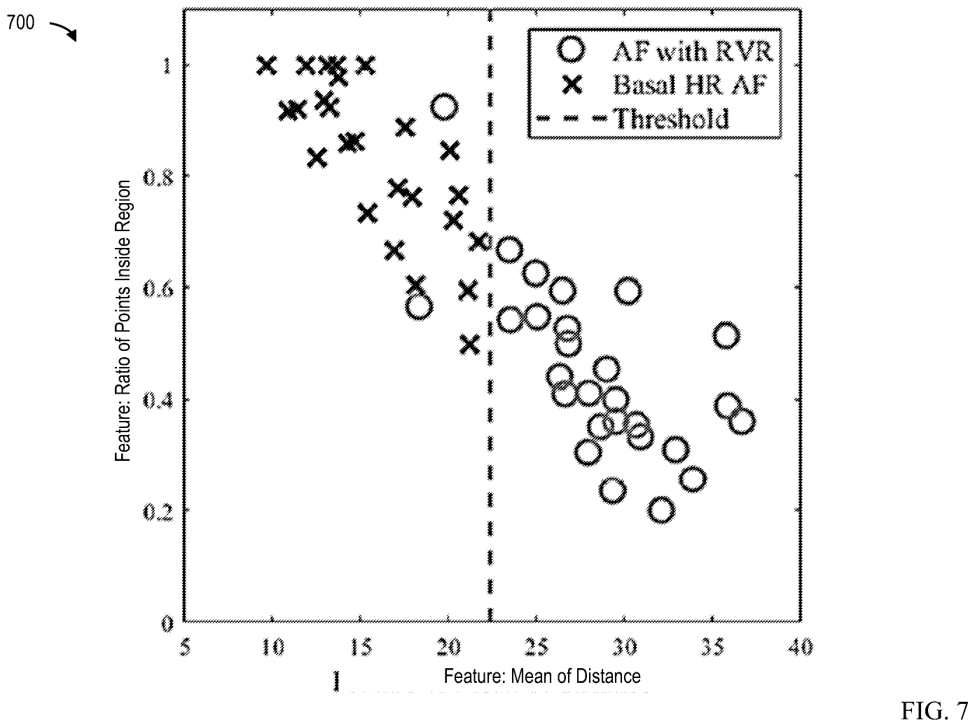

In FIG. 7, a scatter plot 700 of mean distances is shown relative the ratio of points inside the region 608 in accordance with one or more implementations of the present disclosure. If the mean value of distances between points with regard to plots 600, 602 is greater than 22.4 BPM, the PPG signal 126 is considered atrial fibrillation with RVR. If the mean value of distances between points with regard to plots 600, 602 is less than 22.4 BPM, the PPG signal 126 is considered basal atrial fibrillation.

In step 216, for PPG signals 126 corresponding to atrial fibrillation with RVR, a fast beat correction may be implemented. This may necessitate the determination of additional peaks in the PPG signal 126 to address those that may have been missed in other steps. As shown in plots 600, 602, the heart rate difference points are separated into nine quadrants 620, 622, 624, 626, 628, 630, 632, 634, 636. Quadrant 624 represents a fast heartbeat scenario where a fast heartbeat is surrounded by two slow heartbeats, which is indicative of an atrial fibrillation with RVR. It is also possible for undetected heartbeats to occur with data points from the other eight quadrants 620, 622, 626, 628, 630, 632, 634, 636.

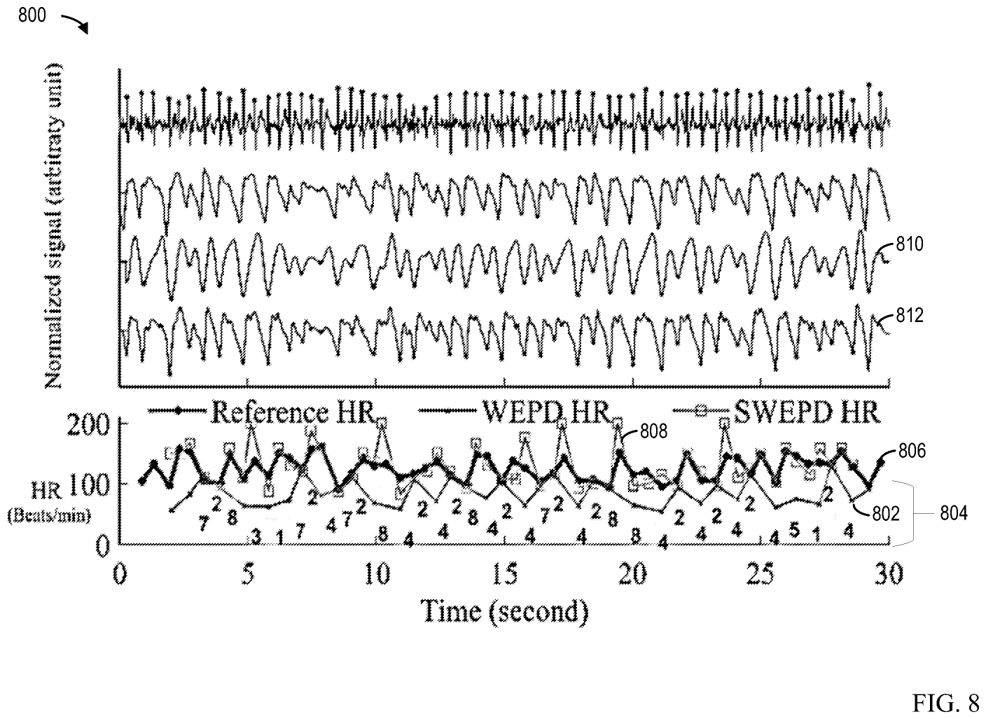

As an example shown in FIG. 8, an example correction for atrial fibrillation with RVR is shown in accordance with one or more implementations of the present disclosure. The heart rates estimated are shown as curve 802 and are associated with quadrants 620, 622, 624, 626, 628, 630, 632, 634, 636 as indicated with numberings 804. As indicated, heartbeats within quadrants 622, 626, 628, 630, 634, 636 deviate from the reference heartbeats and may be corrected. To account for this discrepancy, step 204 may be modified. The band of the bandpass filter discussed herein may be adjusted to allow signals between 0.5 Hz and 10 Hz or otherwise increasing the resulting peaks of the PPG signal 126. Further, beats that are greater than 0.3 seconds apart, (e.g., FIG. 5A) are analyzed for local minima (e.g., local minima 522) to determine additional heartbeats. These fast heartbeat locations at the local minima less than 0.3 seconds apart are combined with the local minima 522 previously found in step 204 to provide indication of all the heartbeats within the PPG signal 126. In such a way, the under performing original WEPD heartbeat estimation in curve 802 is improved to the more accurate SWEPD heartbeat estimation 808 to match the actual heartrate 806. Also shown is a reference ECG, a raw PPG signal, a peak detection from step 204 as WEPD PPG signal 810, and a corrected signal for peak detection of fast heart rates 812.

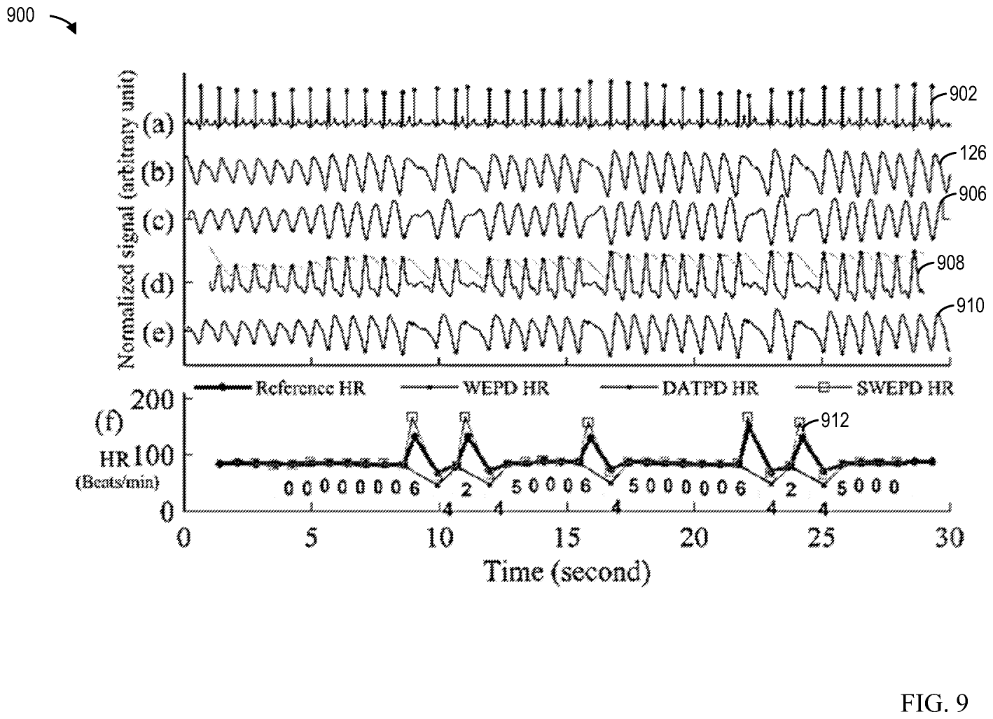

Referring to FIG. 9, an example illustration of PVC and PAC detection is shown in accordance with one or more implementations of the present disclosure. As discussed herein, PAC and PVC detection may reduce false positives and false negatives associated with atrial fibrillation. An example signal of PVC beats 902 is shown. The PPG signal 126 is shown. A peak detection signal 906 from step 204 is shown. A differentiator-adaptive threshold peak detection signal 908 is shown along with PAC and PVC correction as discussed herein.

Such beats are detected fairly well as shown in step 204 but may incorrectly assign some of the beats. A FIR low-pass-differentiator filter with a transition band from 3.5 Hz to 8 Hz may be used to filter the PPG signal 126. This filtration may accentuate the rapid changes in the PPG signal 126 caused by motion and noise, which may lead to erroneous high amplitude peaks in the first derivative step 306 discussed herein. A few steps may be applied to improve peak detection for such a signal. As an example, variable frequency complex demodulation (VFCDM) may be applied to the signal. For example, a modulating frequency may not be fixed but varies as a function of time. For example, Equation 6.

x(t)=dc(t)+A(t)cos(2πf(τ)dτ+ϕ(t) (6)

Multiplying Equation 6 by e^{-j∫}^{₀}^{^{t}}^{2πf(τ)dτ} yields both instantaneous amplitude, A(t), and instantaneous phase, ϕ(t):

z(t)=x(t)e^{-j∫}^{₀}^{^{t}}^{2πf(τ)dτ} (7)

z(t)=−j∫₀^{t}2πf(τ)dτ+A(t)e^{jϕ(t)}+A(t)e^{-j∫}^{₀}^{^{t}}^{4πf(τ)dτ+ϕ(t)} (8)

If z(t) is filtered with an ideal low-pass filter with a cutoff frequency f_{c}<f₀, then the filtered signal zᵢₚ (t) will be obtained with the instantaneous amplitude A(t), and instantaneous phase, ϕ(t). The instantaneous frequency is given by:

$$f ( t ) = {f}_{0} + \frac{1}{2 π} \frac{d ϕ ( t )}{d t} \qquad ( 9 )$$

In the case of variable frequency, the center frequency, f₀, is replaced with a variable frequency. We may first use a center frequency to estimate the instantaneous frequency within the arbitrarily set frequency band using Equation 9. It is reasonable to expect instantaneous frequencies that are changing, especially if the dynamics are highly time varying. An approach may be used that accounts for the possible time varying nature of the instantaneous frequency within the defined frequency bands to obtain a more precise measurement of instantaneous frequency. By changing the center frequency followed by using the variable frequency approach of Equations 6 and 9 along with a low-pass filter, the signal x(t) may be decomposed into the sinusoid modulations by complex demodulation according to:

x(t)=Σᵢdᵢ (10)

x(t)=dc(t)+ΣᵢAᵢ(t)cos(2πf₁(τ)dt+ϕ(t) (11)

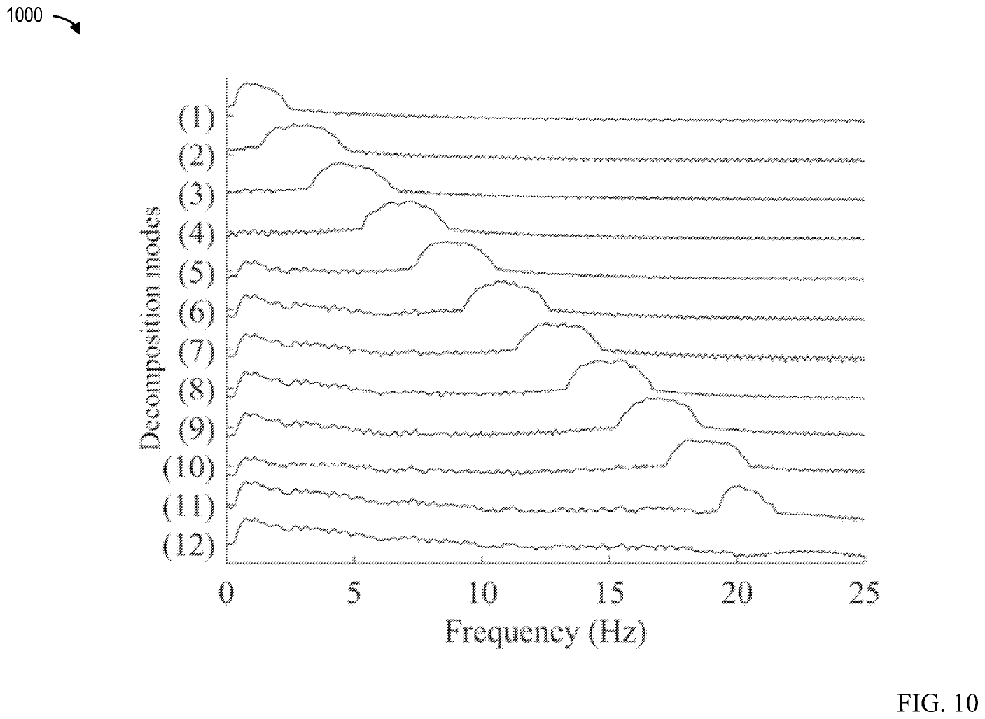

The instantaneous frequency and amplitude of dᵢ can be calculated using the Hilbert transform. The time-frequency spectrum can be obtained by the calculation of the Hilbert transform of Equation 11 from time points for the obtained low-pass filtered frequency components, providing a high resolution time-frequency spectrum and accurate amplitude information. As such, a frequency components from a bandpass filtered PPG signal 126 in the frequency domain is shown in FIG. 10 in accordance with one or more implementations of the present disclosure. It should be appreciated that the first two components are within the heart rate range (e.g., 0.4-4 Hz). The higher components are beyond the heart rate frequency range, thus, are considered to represent higher frequency motion artifact dynamics.

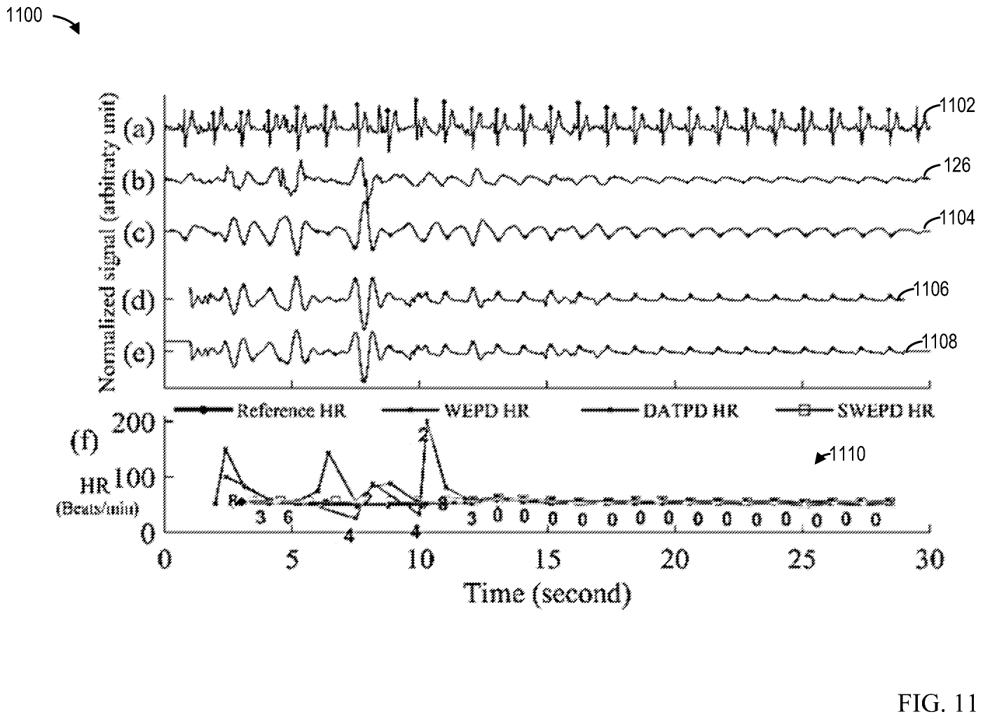

An adaptive threshold may be applied to detect peaks. A refractory period of 300 ms and α=0.2. As an example, FIG. 11 shows an example of the peak correction. A reference ECG 1102 is shown along with raw PPG signal 126. The peak detection signal 1104 from step 204 is also shown. A differentiator-adaptive threshold peak detection signal 1106 is shown along a fast heart rate detection for atrial fibrillation with RVR signal 1108. Curves 1110 are a comparison between the reference heart rate, the estimated heart rates from different methods described herein.

With reference to FIG. 2, fast beat correction is performed in step 232. As an example, PAC and PVC detection may be applied as discussed herein so that their beats are correctly accounted for. It should be appreciated that of the quadrants 620, 622, 624, 626, 628, 630, 632, 634, 636, it is possible that for an improperly detected beat because of PAC or PVC, the next beat will fall within the quadrant 628 of FIG. 6. As an example, beat₁ is likely to be in the quadrant 628 because the difference in the heart rate at n=1 to the prior normal sinus heart rate at n=0 will be relatively large but negative value. In addition, the difference between the heart rate at n=1 to that at the next normal sinus beat (n=2) will be large but a positive value. Thus the beat₁ will be located in quadrant 628. It is likely that when a beat falls within quadrant 628 with a heart rate of less than 60 detected, a PAC or PVC beat has been missed. As such, a bandpass filter between 0.5 Hz and 10 Hz may filter the PPG signal 126 to look for the first minimum point in the time location where the expected PAC or PVC occurred.

It may be necessary to correct for normal sinus rhythms with PAC or PVC. For example, highly deviated heartbeats are compared to either the previous or future heartbeats for examination. As an example, normal sinus rhythm heart beats are mostly confined within the quadrant 620 as there is not much different in heartbeats from one beat to the next. If there is any heartbeat outside quadrant 620 then the average heartbeat from neighboring heartbeats that are within quadrant 620. If all of the heartbeats are outside quadrant 620 then, the mean value of all heart beats within quadrant 620 may be used.

Referring to atrial fibrillation detection in step 206, false positives and false negatives can be pervasive on devices that are mobile, causing motion artifacts and noise. As an example, noise and motion artifacts may cause the PPG signal 126 to become corrupted. As discussed herein, the corrupted windows 128, also referred to as portions of the PPG signal 126, that are corrupted may be correct, ignored, or discarded. As an example, the corrupted windows 128 may be identified and ignored by the atrial fibrillation classification.

As an example, preprocessing of the PPG signal 126 and the acceleration signal 132. As an example, the PPG signal 126 and the acceleration signal 132 may be organized into a window 128. A sixth order Butterworth bandpass filter may be applied. The bandpass filter may have cutoff frequencies of 0.5 and 20 Hz. The PPG signal 126 and acceleration signal 132 may be downsampled. The PPG signal 126 may be downsampled to 50 Hz. The acceleration signal 132 may be downsampled to 30 Hz. The PPG signal 126 and the acceleration signal may be converted to have a set mean (e.g., zero) and unity variance. For example, the acceleration signal 132 may be augmented based on a three-directional axis according to Equation 12.

ACC=\sqrt{aₓ²+a_{y}²+a_{z}²} (12)

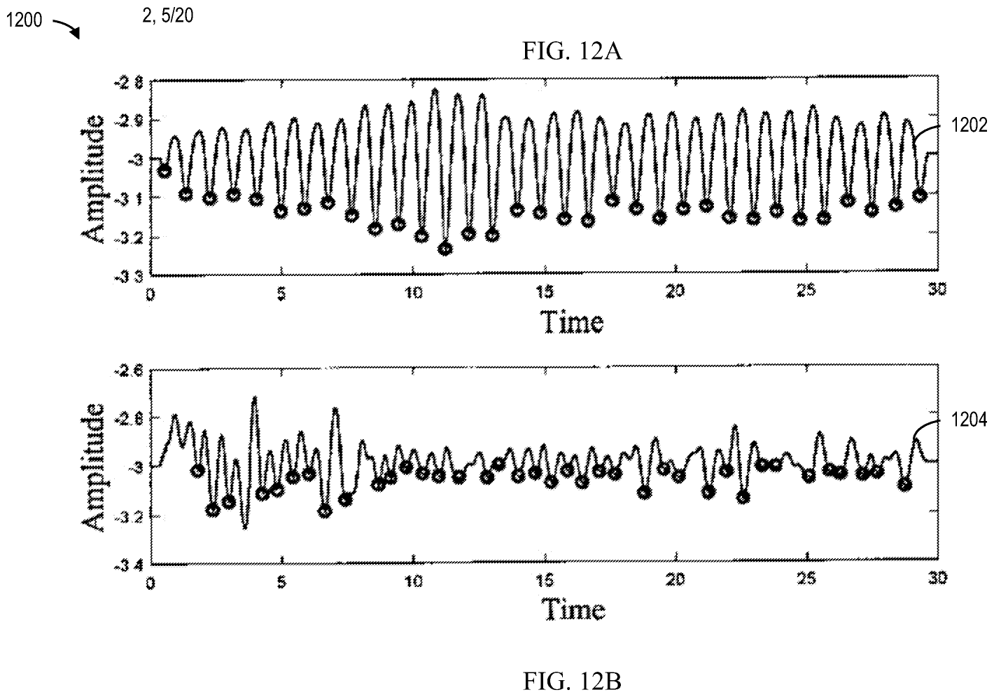

When there is extensive movement, the PPG signal 126 or adjusted PPG signal may be corrupted, as indicated by the acceleration signal 132, as adjusted, or the noise within the PPG signal 126. In FIG. 12A, a clean PPG signal 1202 is shown based on the PPG signal 126, and in FIG. 12B a corrupted PPG signal 1204 is shown based on the PPG signal 126 in accordance with one or more implementations of the present disclosure.

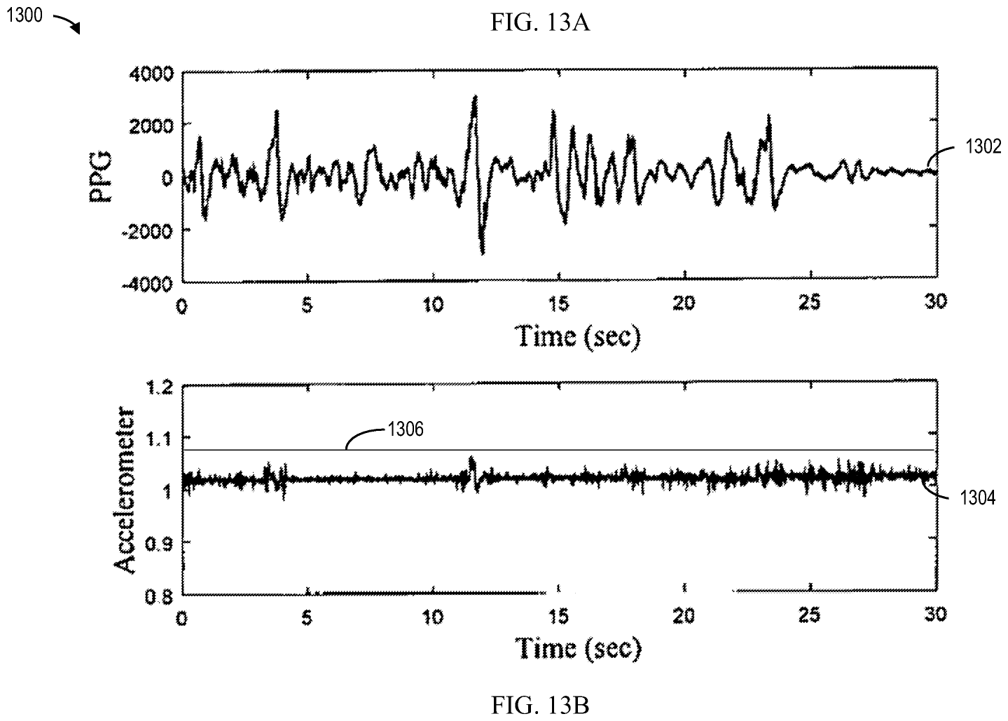

In FIG. 13, a PPG signal 126 that is corrupted and an acceleration signal 132 is shown in accordance with one or more implementations of the present disclosure. As the variance increases due to motion artifacts, the mean value of the acceleration signal 132 becomes higher for a corrupted segment while the mean value is lower for a clean segment. Since the converted acceleration signal is of unity variance, the mean value of the acceleration signal 132, now converted, may become lower for a corrupted segment while the mean value is higher for a clean segment. A value indicative of corruption 1302 associated with the acceleration signal 132 may indicate when the acceleration signal 1304 is indicative of a PPG signal 126 that is corrupted. As such, the instant window 128 may be discarded or ignored upon the acceleration signal 132 exceeding the value indicative of corruption 1302.

In some circumstances, including that shown in FIG. 13A, a corrupted PPG signal 1302 may be present without indication from the acceleration signal, shown in FIG. 13B, due to noise, which may lead to false positive determinations of atrial fibrillation.

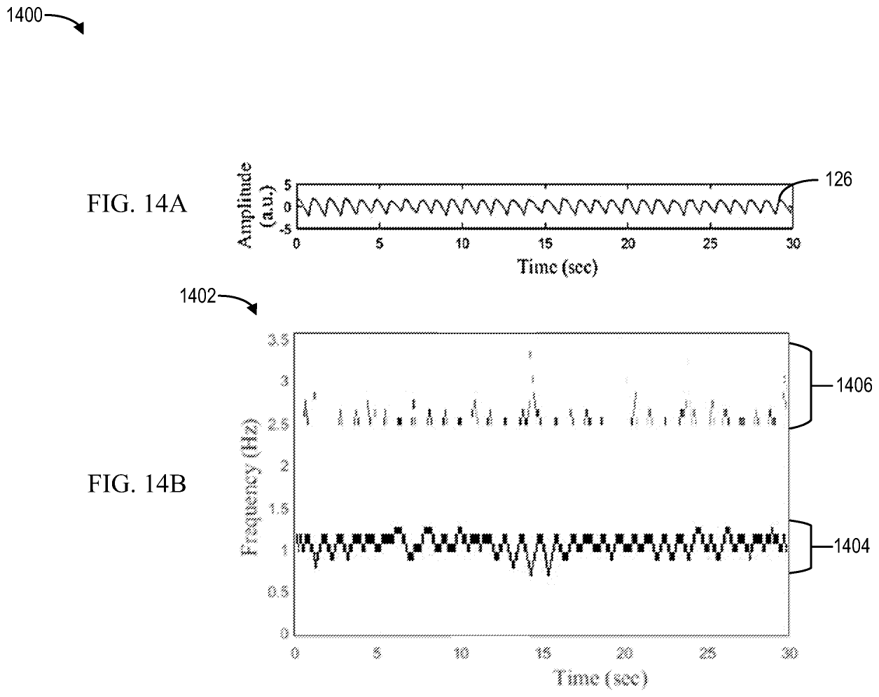

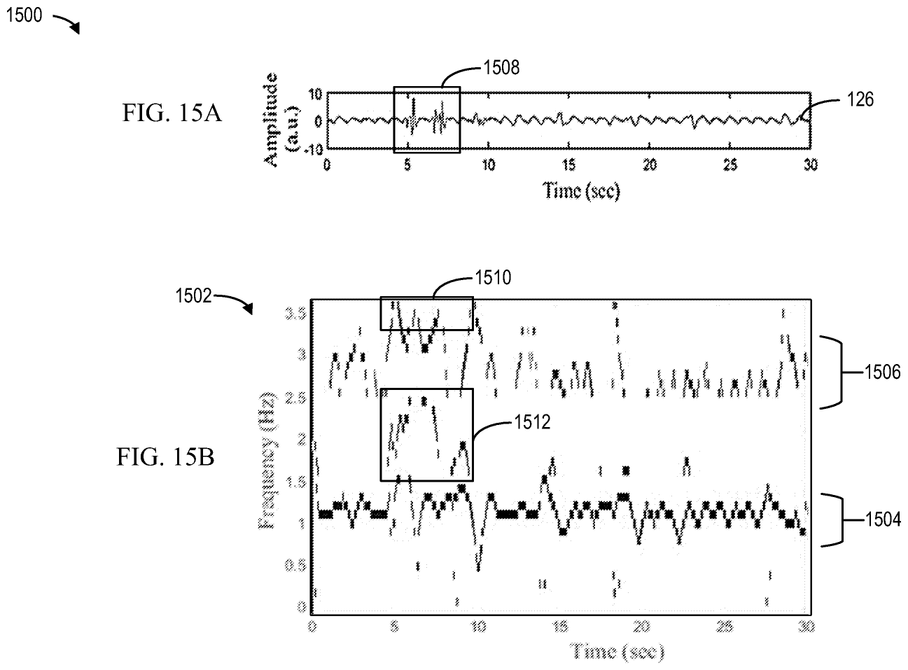

In FIG. 14B, a time-frequency spectrum 1400 is shown with respect to the PPG signal 126, as shown in FIG. 14A, in accordance with one or more implementations of the present disclosure. VFCDM may be used to break down the frequency components of the PPG signal 126 forming a time-frequency spectra 1402. The time-frequency spectra 1402 depicts energies of frequencies within the PPG signal 126 over time. As an example, the frequency, on the Y-axis, may be broken into components or ranges. As shown with a clean PPG signal 126 the frequency components have energies that fall within two different components 1404 and 1406. The primary frequency component 1404 may characterize energies within a band between 0.75 Hz and 1.5 Hz. The secondary frequency component 1406 may characterize energies within a band between 2.5 and 3 Hz. These are the dominant frequency components. When there is noise or motion artifacts present, there is a cognizable change in the time-frequency spectra 1402. As an example, shown in FIG. 15, a noisy time-frequency spectrum 1502 is shown in accordance with one or more implementations of the present disclosure.

As shown, the time-frequency spectrum 1502 has frequency components 1504, 1506 identified between the bands discussed above. Because of the noise 1508 interjected in signal 126 the time-frequency spectrum 1502 includes outside of the bands discussed above. As an example, the noise is represented in areas 1510, 1512. Instructions may be executed by the processor 108 to detect these energies that are less than frequencies of the primary frequency component 1504, greater than frequencies of the primary frequency component 1504 and less than frequencies of the secondary frequency component 1506, greater than frequencies of the secondary frequency component 1506, or combinations thereof. As such, noise 1508 may be detected within the signal 126 to determine whether the window 128 shown is clean or noisy.

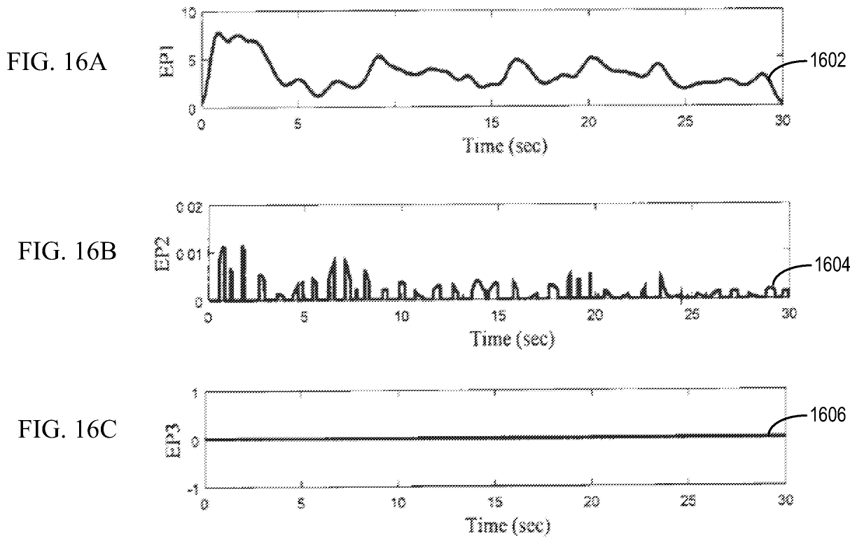

As an example, the quantified energy within the frequency components is shown in FIGS. 16A-16C in accordance with one or more implementations of the present disclosure. For instance, the quantified energy of the primary frequency component 1504 is shown as curve 1602 over time. The quantified energy of the secondary frequency component 1506 is shown as curve 1604 over time. The second frequency component may be orders of magnitude smaller than the primary frequency component 1504. As another example, the quantified energy of the other frequency components, or those outside of the primary frequency component 1504 and the secondary frequency component 1506 is shown as curve 1604, which may be barely detectable when noise and motion artifacts are not present.

To quantify this property, from the time-frequency spectra 1402 in the heart rate range, counts of these energy quantities may be determined, which may be used to distinguish PPG signals 126 that are clean from PPG signals 126 that are noisy. As indicated in Equations 13 and 14, the presence of these energies may be quantified.

Q₁=count of energy of the first two dominant frequency components (13)

Q₂=count of energy of the remaining frequency components (14)

A clean signal may be indicated when Q₁ is substantially high and Q₂ is substantially low. As an example, the time instances of Q₁ and Q₂ may be counted according to a count period. If energy over an energy value is present in the first frequency component 1504, the second frequency component 1506, or a combination of the first frequency component 1504 and the second frequency component 1506, Q₁ may be incremented by one or more. The energy value may be different for the first frequency component 1504 and the second frequency component 1506. If energy over another energy value is present, which may be similar or different from the energy value, in the other frequency component as shown in curve 1606, Q₂ may be incremented by one or more. As such, these occurrences may be tabulated over the entire window 128. If Q₁ is greater than a tabulation quantity associated with the first frequency component 1504 and the second frequency component 1506 and Q₂ is less than a tabulation quantity associated with the other frequency components, the window 128 may be determined to be clean. Otherwise, the window 128 may be determined to be noisy.

As an example, a window 128 may be determined noisy if the acceleration signal 132 is indicative of corruption, based on the value indicative of corruption 1306. The window 128 may also be determined noisy based on the first frequency component 1504, the second frequency component 1506, and the other frequency components. In such a way, atrial fibrillation may be detected on clean signals that are verified according to the acceleration signal 132, the frequency components of the PPG signal 126, and combinations thereof.

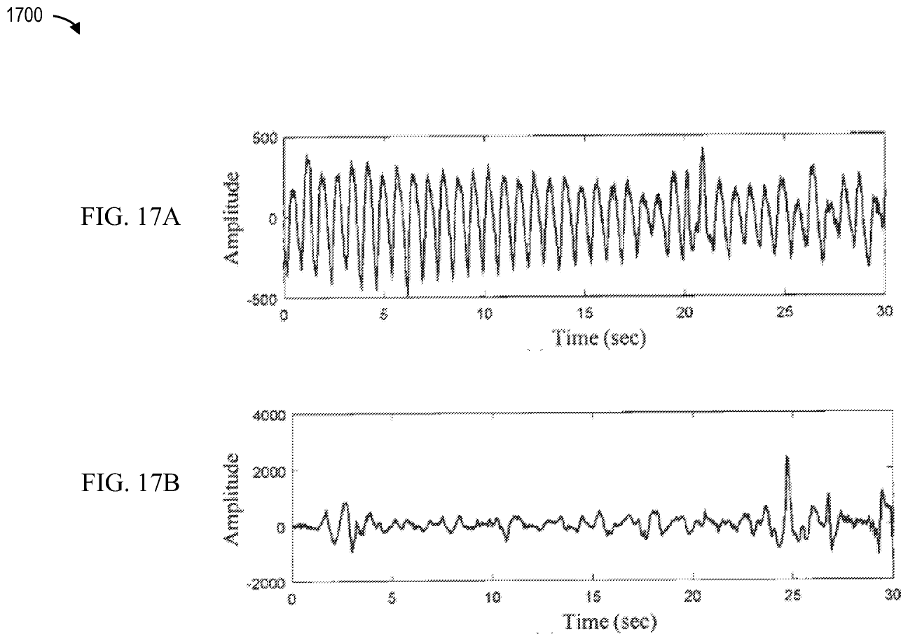

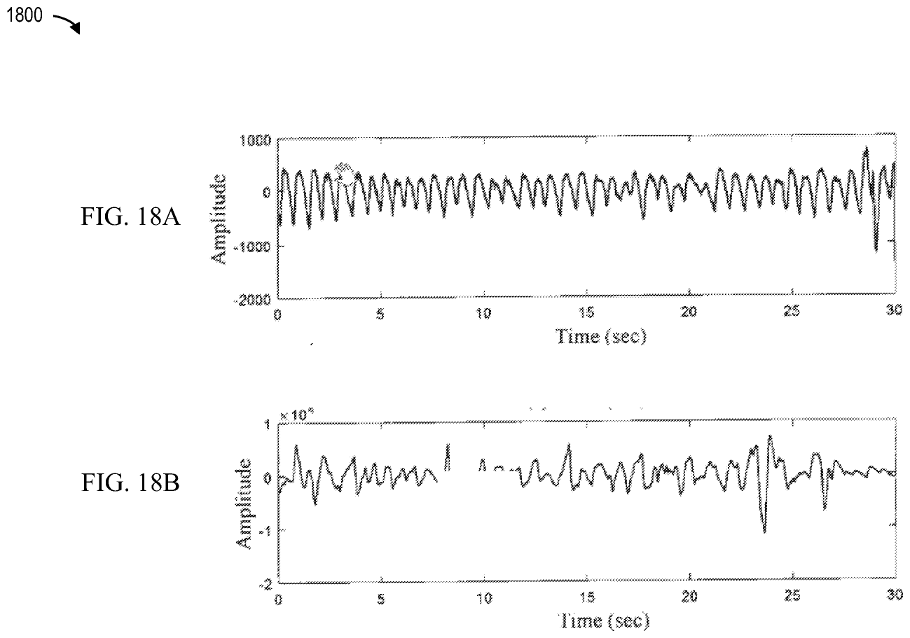

In FIG. 17A, a PPG signal 126 over a window 128 is shown that is detected as clean, with normal sinus rhythm, in accordance with one or more implementations of the present disclosure. In FIG. 17B, a PPG segment 126 over a window 128 is shown that is detected as corrupted, with normal sinus rhythm, in accordance with one or more implementations of the present disclosure. In FIG. 18A, a PPG signal 126 over a window 128 is shown that is detected as clean with atrial fibrillation in accordance with one or more implementations of the present disclosure. In FIG. 18B, a PPG segment 126 over a window 128 is shown that is detected as corrupted, with atrial fibrillation, in accordance with one or more implementations of the present disclosure.

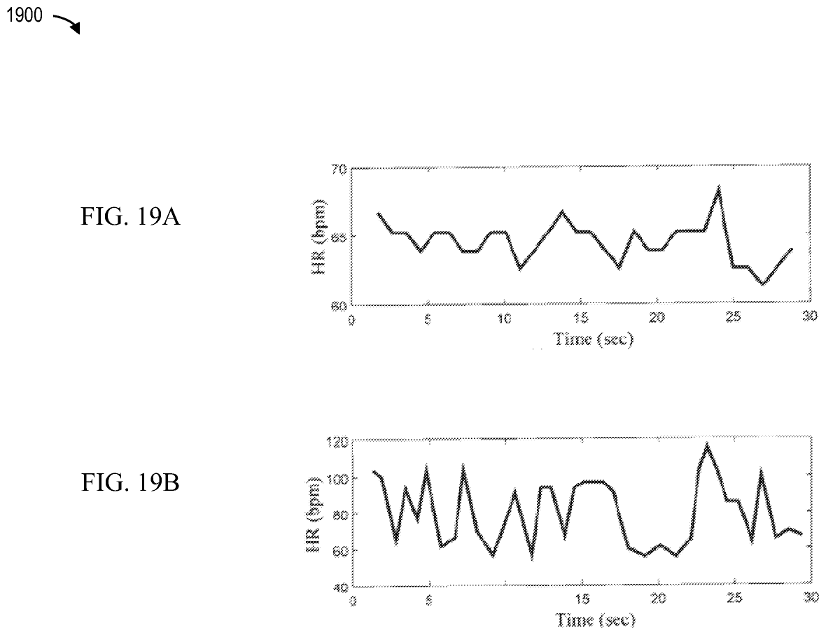

A window 128 that has been determined clean, or another window, atrial fibrillation detection may be performed. Atrial fibrillation may be defined as an irregularly irregular heart rate. As shown in FIG. 19A, a substantially constant heart rate is shown over time that depicts normal sinus rhythm in accordance with one or more implementations of the present disclosure. As shown in FIG. 19B, an irregularly irregular heart rate is shown over time that depicts atrial fibrillation in accordance with one or more implementations of the present disclosure.

A root mean square of successive differences (RMSSD) calculation may be performed along with sample entropy from the pulse intervals corresponding to the window 128. As such, the beat-to-beat variability may be determined by the RMSSD and high variability may be associated with atrial fibrillation, as shown in Equation 15.

RMSSD={square root over (Σₜ₌₁^{l=1}(x(i+1)−x(i))²)} (15)

where x(i) is a sequence of peak-to-peak or beat-to-beat, intervals of length l. The RMSSD may be divided by the mean of the peak-to-peak time series as it may be more consistent than using RMSSD of the time series alone because the variability and mean of heart rate values differ from subject to subject and segment to segment. Sample entropy, SampEn, may also be calculated, and used, to determine atrial fibrillation.

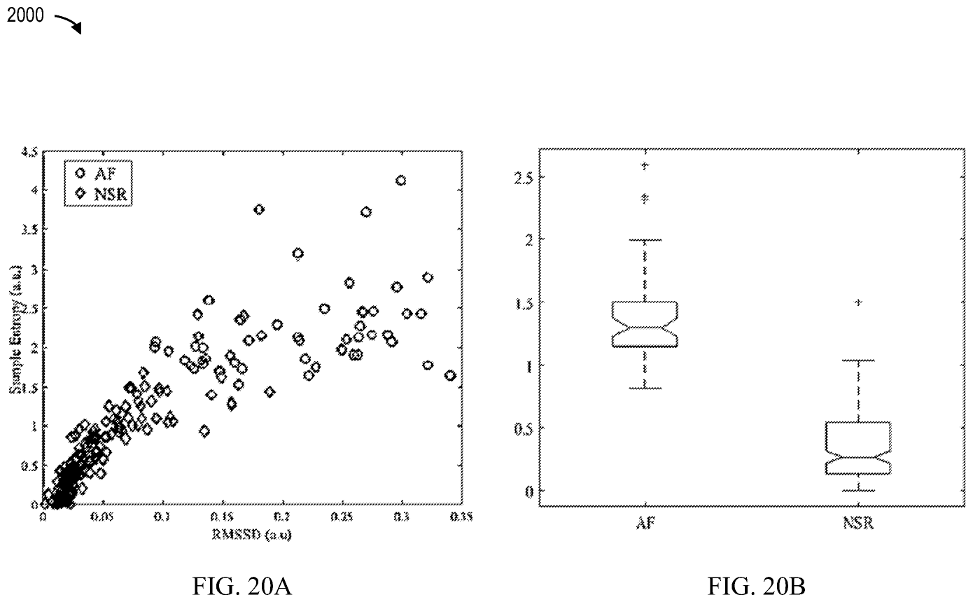

In FIG. 20A, a plot is shown indicating relative placements between windows of SampEn with respect to RMSSD. And in FIG. 20B, a box plot indication of the respective atrial fibrillation determination is also presented. As an example, the processor 108, or another implement, may determine the atrial fibrillation from a PPG signal 126 based on a combination of RMSSD and SampEn as shown in Equation 16.

Combination=w*RMSSD+(1−w)*SampEn (16)

where w is a weighting factor between RMSSD and SampEn. If the calculated weighted combination of RMSSD and SampEn is greater than a combination value (e.g., 0.94) atrial fibrillation is detected and treatment can be administered. Secondary screening of atrial fibrillation may be performed as is discussed herein.

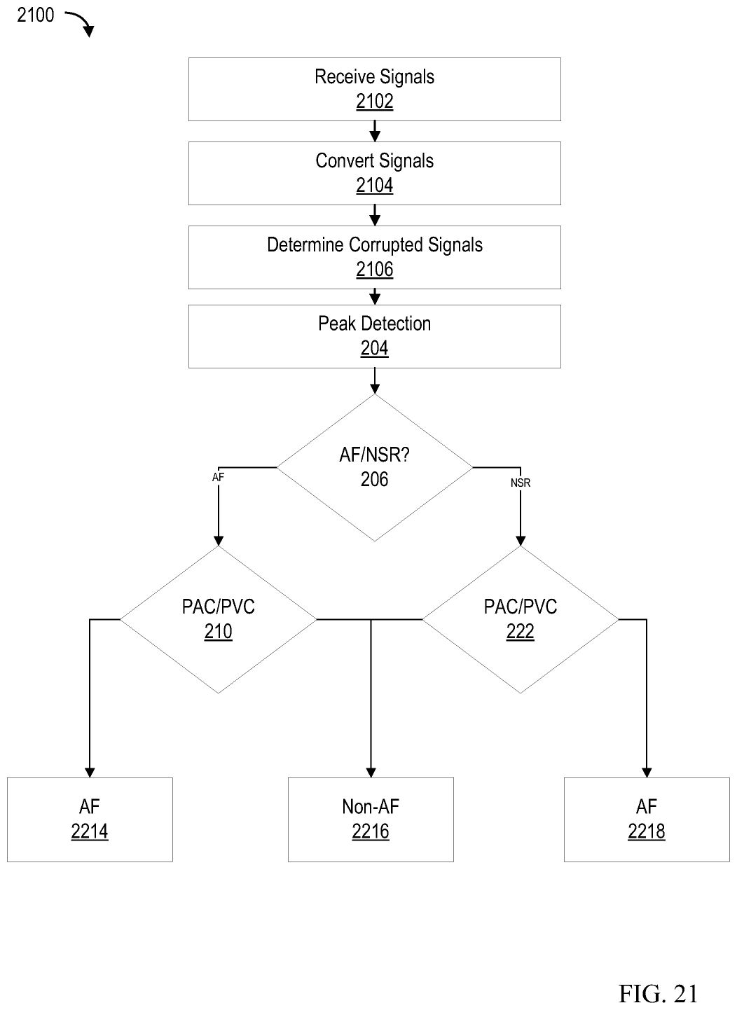

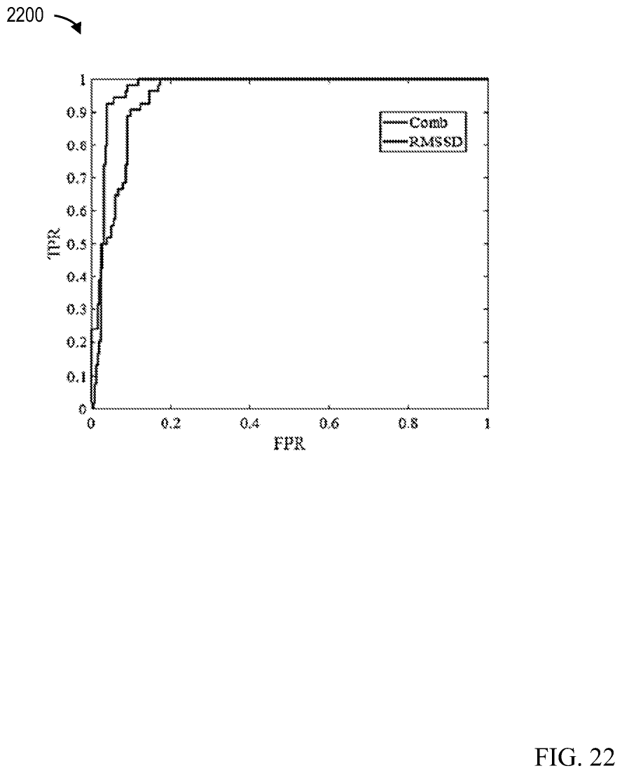

In FIG. 21, an example method 2100 for determining atrial fibrillation is shown in accordance with one or more implementations of the present disclosure. In step 2102, the PPG signal 126 and the acceleration signal 132 are received. In step 2104, the PPG signal 126 and the acceleration signal 132 are converted, which may include normalization, as described herein. In step 2106, windows 128 are determined to be corrupted or clean based on the PPG signal 126, the acceleration signal 132, other signals and combinations thereof as is described throughout this disclosure. In step 204, peak detection as described herein may be performed on the windows 128 that are designated as clean or other windows. In step 206, the determination of atrial fibrillation is performed. This determination may be based on the combination of RMSSD and SampEn as discussed herein. As discussed, steps 210, 222 may distinguish atrial fibrillation and normal sinus rhythm determinations from PAC and PVC into final categories of atrial fibrillation in steps 2214 and non-atrial fibrillation in step 2216. As such, treatments may be administered based on these determinations. As shown, such determinations may improve the treatment of atrial fibrillation by ensure the proper administration of such treatments with less unnecessary treatments and less missed potential treatments as indicated in FIG. 22 in accordance with one or more implementations of the present disclosure.

PAC and PVC are generally benign cardiac arrhythmias that occur in some healthy subjects; their impact on overall cardiovascular well-being is still unclear. As irregular beats can be interspersed in PPG signals 126 having PAC and PVC characteristics, this can lead to false positives of atrial fibrillation. As an example, PAC and PVC detection may be performed on both PPG data obtained from the fingertip and PPG data obtained from the wrist. PPG signals 126 that are obtained from the wrist may be more difficult to distinguish because the skin is thicker and motion artifacts on the wrist may be less severe than motion artifacts from the fingertip.

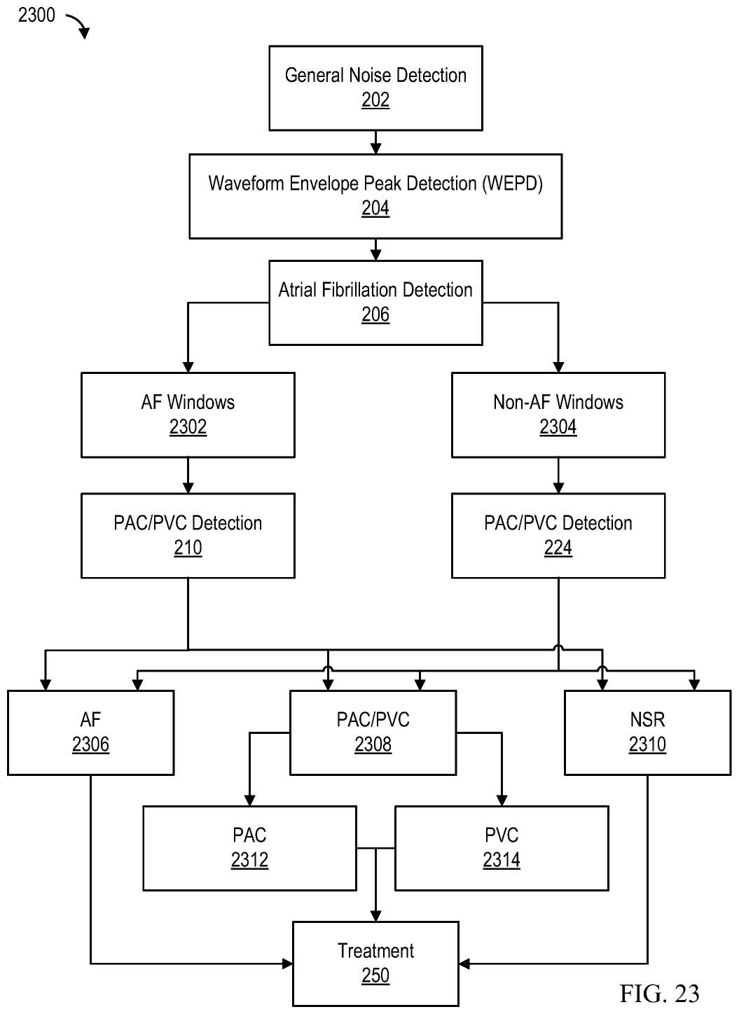

Referring to FIG. 23, a method 2300 for PVC and PAC determination is shown in accordance with one or more implementations of the present disclosure. In step 202, general noise detection is performed as discussed herein along with waveform envelope peak detection (WEPD) in step 204 and atrial fibrillation determinations in step 206. PAC and PVC determinations may be performed on windows 128 associated with atrial fibrillation and normal sinus rhythms.

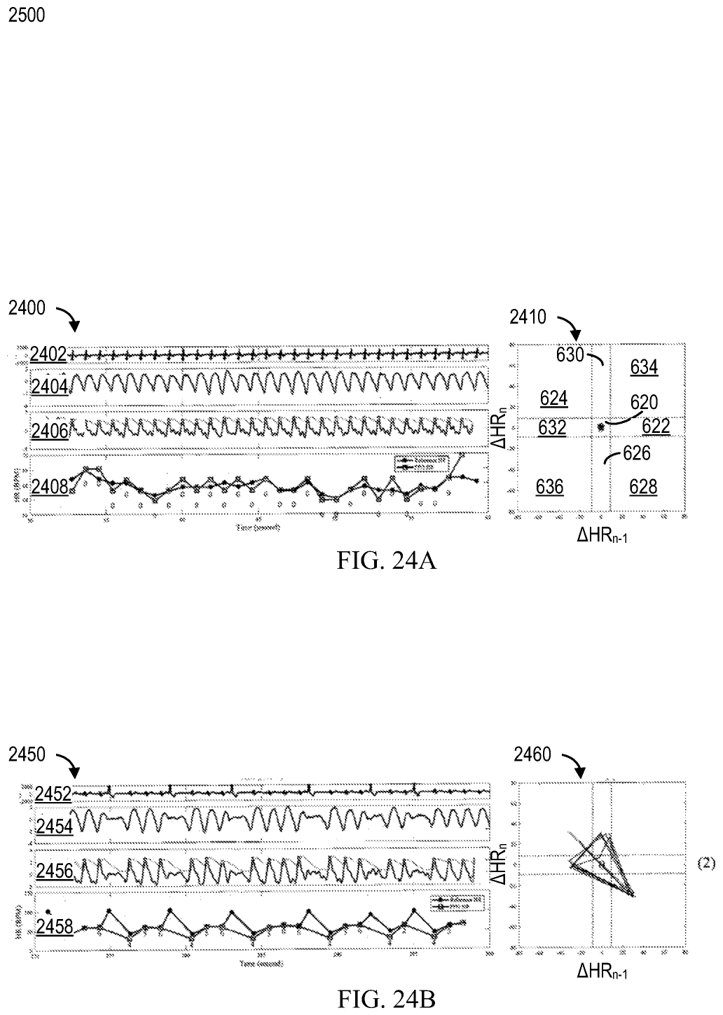

In FIG. 24A, a normal sinus rhythm (NSR) panel 2400 is shown over a window 128 (e.g., 30 seconds) in accordance with one or more implementations of the present disclosure. An NSR ECG 2402 is shown with respect to a NSR PPG signal 2404 along with a NSR peak detected PPG signal 2406. In box 2408, a reference heart rate is shown alongside a predicted heart rate. A respective Poincare plot 2410 indicates the recurrence of a previous heart rate difference (e.g., current beat to the previous beat) and a current heart rate difference (e.g., current beat to the next beat) over time. As shown in plot 2410, relatively little difference occurs with respect to the NSR PPG signal 2404 and the entire plot is within quadrant 620. It should be appreciated that although shown as a Poincare plot, processor 108 may be operable upon execution of instructions to determine the points of the plot 2410 without vision representation.

In FIG. 24B, a PVC panel 2450 is shown over a window 128 (e.g., 30 seconds) in accordance with one or more implementations of the present disclosure. A PVC ECG 2452 is shown with respect to a PVC PPG signal 2454 along with a PVC peak detected PPG signal 2456. In box 2458, a reference heart rate is shown alongside a predicted heart rate. A respective Poincare plot 2460 indicates the recurrence of a previous heart rate difference (e.g., current beat to the previous beat) and a current heart rate difference (e.g., current beat to the next beat) over time. As shown in plot 2460, a kite shape is formed based on the previous heart rate difference and the current heart rate difference. The scale of the X-axis and Y-axis is −80 to 80 BPM. As an example, the Poincare plot as divided into nine quadrants 620, 622, 624, 626, 628, 630, 632, 634, 636, indicating long and short time differences based on the three consecutive heart beat intervals. Although shown as dividing quadrants at [−10, 10] heartrate difference, various segmentations may be used (e.g., [−10, 20] and [−20, 20]). A long time difference may occur when a time difference of the heartbeats associated with either the PAC or PVC beat to the next normal heartbeat is compared to the time difference between two consecutive normal heartbeats. A short time difference may occur when a time difference between a previous normal beat to either a PAC or PVC beat is compared to the time difference between two consecutive normal heartbeats. It should be appreciated that although shown as a Poincare plot, processor 108 may be operable upon execution of instructions to determine the points of the plot 2410 without vision representation.

To distinguish between atrial fibrillation, PVC, PAC, and NSR a lookup table may be used. An example of quadrants associated with the three consecutive heartbeats is shown in Table 1.

TABLE 1

| HRₙ₋₁-HRₙ-HRₙ₊₁ | Quadrant Number |
|---|---|
| slow-slow-slow | 0 (e.g., quadrant 620) |
| fast-fast-fast | 0 |
| slow-slow-fast | 1 (e.g., quadrant 622) |
| slow-fast-slow | 2 (e.g., quadrant 624) |
| fast-slow-slow | 3 (e.g., quadrant 626) |
| fast-slow-fast | 4 (e.g., quadrant 628) |
| slow-fast-fast | 5 (e.g., quadrant 630) |
| fast-fast-slow | 6 (e.g., quadrant 632) |
| slow-fast-ultra fast | 7 (e.g., quadrant 634) |
| ultra fast-fast-slow | 8 (e.g., quadrant 638) |

An example of PVC and PAC patters is shown in Table 2.

TABLE 2

| Types of PAC/PVC | Trajectory Patterns |
|---|---|
| Bigeminy (normal heartrate) | 2-4-2-4-2-4 |
| Bigeminy (fast heartrate) | 0-0-0-0-0-0 |
| Trigeminy (fast heartrate) | 2-4-2-4-2-4 |
| Trigeminy (normal heartrate) | 1-2-3-1-2-3 |
| Quadrigeminy (normal heartrate) | 6-4-5-6-4-5 |
| Quadrigeminy (normal heartrate) | 1-2-3-0-1-2-3 |

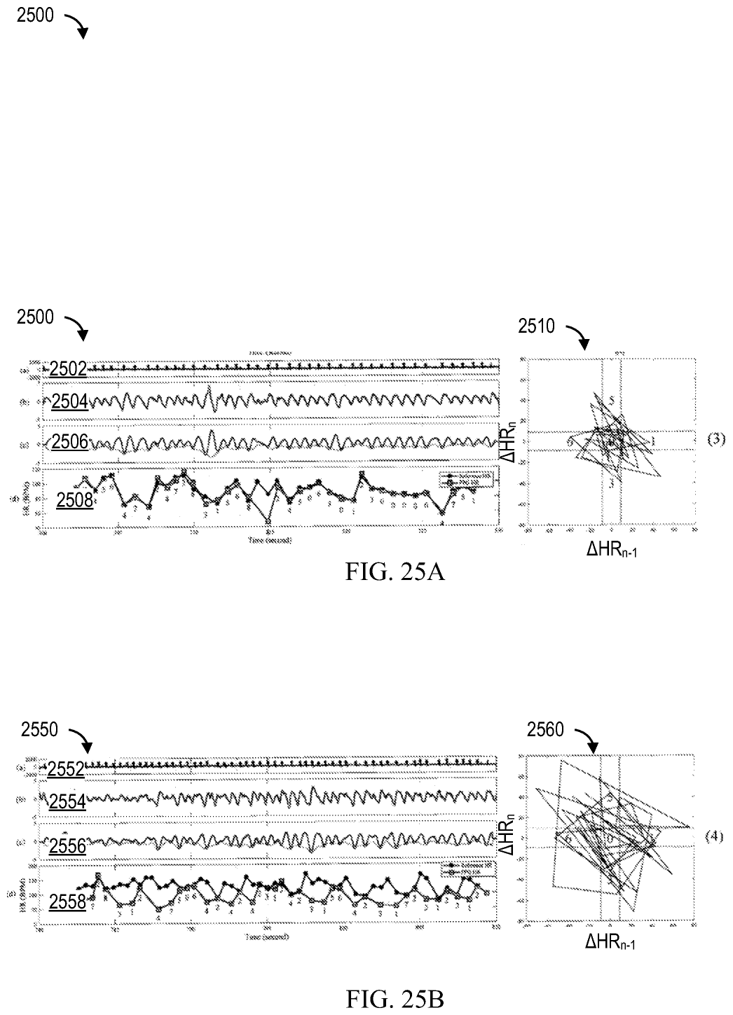

In FIG. 25A, a normal heart rate atrial fibrillation (NAF), or basal heart rate atrial fibrillation, panel 2500 is shown over a window 128 (e.g., 30 seconds) in accordance with one or more implementations of the present disclosure. An NAF ECG 2402 is shown with respect to a NAF PPG signal 2504 along with a NAF peak detected PPG signal 2506. In box 2508, a reference heart rate is shown alongside a predicted heart rate. A respective Poincare plot 2510 indicates the recurrence of a previous heart rate difference (e.g., current beat to the previous beat) and a current heart rate difference (e.g., current beat to the next beat) over time. As shown in plot 2510, drastic difference occurs with respect to the NAF PPG signal 2504.

In FIG. 25B, a fast heart rate atrial fibrillation (FAF) panel 2550 is shown over a window 128 (e.g., 30 seconds) in accordance with one or more implementations of the present disclosure. An FAF ECG 2552 is shown with respect to a FAF PPG signal 2554 along with a FAF peak detected PPG signal 2556. In box 2558, a reference heart rate is shown alongside a predicted heart rate. A respective Poincare plot 2560 indicates the recurrence of a previous heart rate difference (e.g., current beat to the previous beat) and a current heart rate difference (e.g., current beat to the next beat) over time. As shown in plot 2560, drastic difference occurs with respect to the FAF PPG signal 2554.

PAC and PVC may be determined according to ΔHRₙ₋₁ and ΔHRₙ as trigeminy PAC or PVC when patterns are in quadrants 622, 624, 626, defining a “1-2-3” pattern. Bigeminy PAC or PVC may be determined according to ΔHRₙ₋₁ and ΔHRₙ having points in quadrants 624, 628, defining a “2-4” pattern. Additionally, a fast heart rate PAC and PVC may be determined when patters are in quadrants 628, 630, 632, defining a “6-4-5” pattern. As an example, three adjacent heart rates may be used to determine an initial point on plot 2460. This point may be located in quadrant 632 (e.g., [−40 ΔHRₙ, 5 ΔHRₙ₋₁]). The next adjacent heart rates, which may include two heart rates from the previous ΔHRₙ₋₁ and ΔHRₙ calculation, may be used to determine another point located in quadrant 628. And another set of adjacent heart rates, which may include one of the heart rates from the first ΔHRₙ₋₁ and ΔHRₙ calculation, may be used to determine the last point located in quadrant 630. A determination of atrial fibrillation may be improved using these indications.

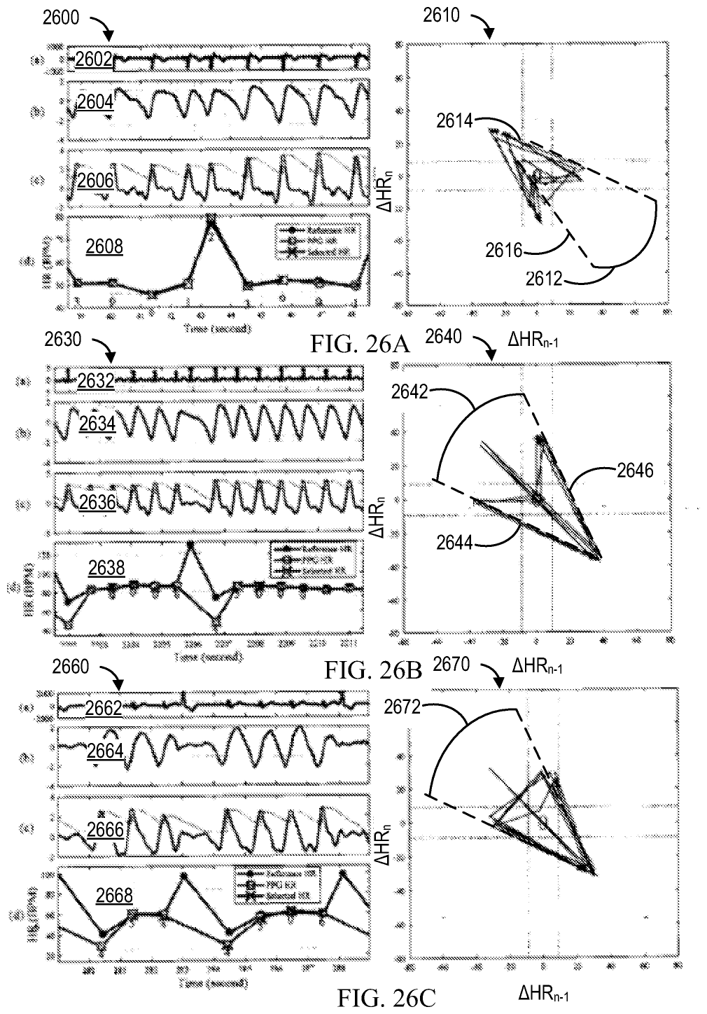

In FIG. 26A, a 1-2-3 pattern panel 2600 for normal heart rate PAC is shown over a window 128 (e.g., 30 seconds) in accordance with one or more implementations of the present disclosure. A 1-2-3 pattern ECG 2602 is shown with respect to a 1-2-3 pattern PPG signal 2604 along with a 1-2-3 pattern peak detected PPG signal 2606. In box 2608, a reference heart rate is shown alongside a predicted heart rate. A respective Poincare plot 2610 indicates the recurrence of a previous heart rate difference (e.g., current beat to the previous beat) and a current heart rate difference (e.g., current beat to the next beat) over time. As shown in plot 2610, drastic difference occurs with respect to the 1-2-3 pattern PPG signal 2604.

In FIG. 26B, a 6-4-5 pattern panel 2630 for fast heart rate PAC is shown over a window 128 (e.g., 30 seconds) in accordance with one or more implementations of the present disclosure. A 6-4-5 pattern for fast heart rate PAC ECG 2632 is shown with respect to a 6-4-5 pattern for fast heart rate PAC PPG signal 2634 along with a 6-4-5 pattern for fast heart rate PAC peak detected PPG signal 2636. In box 2638, a reference heart rate is shown alongside a predicted heart rate. A respective Poincare plot 2640 indicates the recurrence of a previous heart rate difference (e.g., current beat to the previous beat) and a current heart rate difference (e.g., current beat to the next beat) over time. As shown in plot 2640, drastic difference occurs with respect to the 6-4-5 pattern for fast heart rate PAC PPG signal 2634.

In FIG. 26C, a 6-4-5 pattern panel 2660 for fast heart rate PVC is shown over a window 128 (e.g., 30 seconds) in accordance with one or more implementations of the present disclosure. A 6-4-5 pattern for fast heart rate PVC ECG 2662 is shown with respect to a 6-4-5 pattern for fast heart rate PVC PPG signal 2664 along with a 6-4-5 pattern for fast heart rate PVC peak detected PPG signal 2666. In box 2668, a reference heart rate is shown alongside a predicted heart rate. A respective Poincare plot 2670 indicates the recurrence of a previous heart rate difference (e.g., current beat to the previous beat) and a current heart rate difference (e.g., current beat to the next beat) over time. As shown in plot 2670, drastic difference occurs with respect to the 6-4-5 pattern for fast heart rate PVC PPG signal 2664.

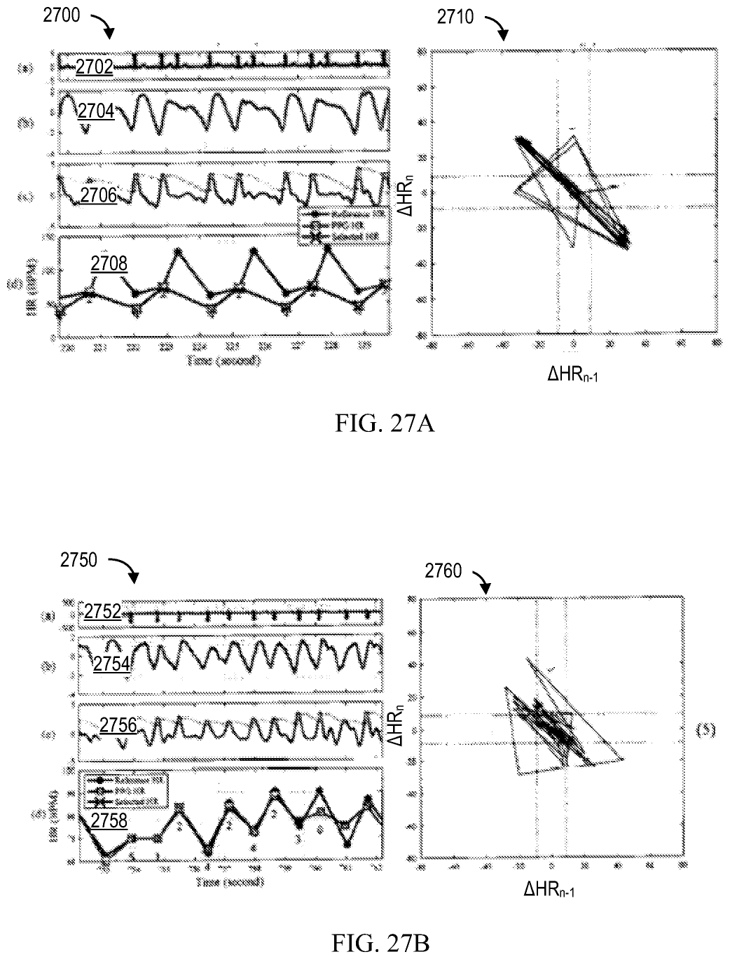

In FIG. 27A, a 2-4 pattern panel 2700 for fast heart rate PAC with trigeminy is shown over a window 128 (e.g., 30 seconds) in accordance with one or more implementations of the present disclosure. A 2-4 pattern for fast heart rate PAC with trigeminy ECG 2702 is shown with respect to a 2-4 pattern for fast heart rate PAC with trigeminy PPG signal 2704 along with a 2-4 pattern for fast heart rate PAC with trigeminy peak detected PPG signal 2706. In box 2708, a reference heart rate is shown alongside a predicted heart rate. A respective Poincare plot 2710 indicates the recurrence of a previous heart rate difference (e.g., current beat to the previous beat) and a current heart rate difference (e.g., current beat to the next beat) over time. As shown in plot 2710, drastic difference occurs with respect to the 2-4 pattern for fast heart rate PAC with trigeminy PPG signal 2704.

In FIG. 27B, a 2-4 pattern panel 2750 for fast heart rate PAC with bigeminy is shown over a window 128 (e.g., 30 seconds) in accordance with one or more implementations of the present disclosure. A 2-4 pattern for fast heart rate PAC with bigeminy ECG 2752 is shown with respect to a 2-4 pattern for fast heart rate PAC with bigeminy PPG signal 2754 along with a 2-4 pattern for fast heart rate PAC with bigeminy peak detected PPG signal 2756. In box 2758, a reference heart rate is shown alongside a predicted heart rate. A respective Poincare plot 2760 indicates the recurrence of a previous heart rate difference (e.g., current beat to the previous beat) and a current heart rate difference (e.g., current beat to the next beat) over time. As shown in plot 2760, drastic difference occurs with respect to the 2-4 pattern for fast heart rate PAC with bigeminy PPG signal 2754.

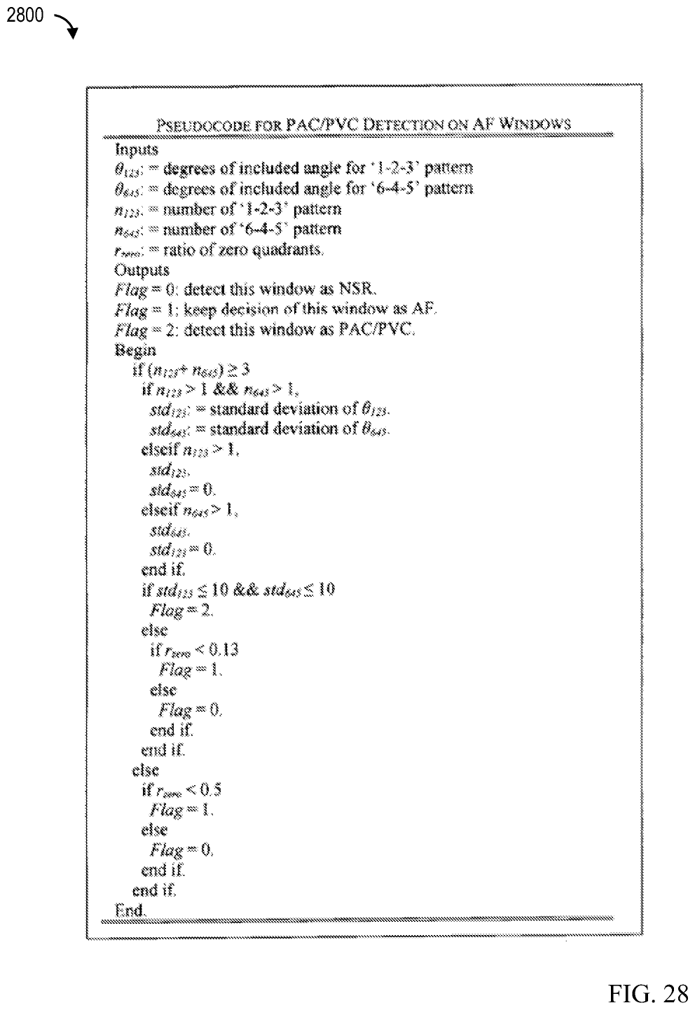

In FIG. 28, pseudocode 2800 for PAC and PVC detection of atrial fibrillation windows is shown in accordance with one or more implementations of the present disclosure. As discussed with respect to step 210, the window 128 may have been determined to present indications of atrial fibrillation in steps 206, 2402. As discussed herein, the values of ΔHRₙ₋₁ and ΔHRₙ are calculated to define coordinates [ΔHRₙ₋₁, ΔHRₙ]. A count may be taken to determine the quantity of heart rate differences within particular quadrants 620, 622, 624, 626, 628, 630, 632, 634, 636. As an example, n₁₂₃ corresponds to a sequence of coordinates that fall within the quadrants 622, 624, 626, or another order of the quadrants 622, 624, 626 (e.g., 622, 626, 624). n₆₄₅ corresponds to a sequence of coordinates that fall within the quadrants 632, 628, 630, or another order of the quadrants 632, 628, 630 (e.g., 628, 630, 632). As also indicated in FIGS. 26A-C an angle 2612, 2642, 2672 may be determined for coordinates within n₁₂₃ and n₆₄₅ and a ratio between the quantity of coordinates falling within quadrant 620 and the total quantity of coordinates. The algorithm may use these values to determine whether the windows 128 is a normal sinus rhythm, atrial fibrillation, PAC, or PAC. As an example, the combination of n₁₂₃, n₆₄₅, θ₁₂₃, θ₆₄₅, and r_{zero} can be used to determine NSR, PAC, PVC, and atrial fibrillation as shown. Vector resemblance may be used to represent the similarity of the PAC and PVC trajectories. As an example, θ₁₂₃ is equal to the 1-2-3 angle 2602 formed between a 1-2 vector 2614 between a point in quadrant 622 and a point in quadrant 624 and a 2-3 vector 2616 between the point in quadrant 624 and a point in quadrant 626. As another, θ₆₄₅ is equal to the 6-4-5 angle 2642 formed between a 6-4 vector 2634 between a point in quadrant 632 and a point in quadrant 628 and a 4-5 vector 2616 between the point in quadrant 628 and a point in quadrant 630.

A standard deviation, std₁₂₃, of θ₁₂₃ over the window 128 is calculated along with std₆₄₅, of θ₆₄₅ over the window 128 to sort out similar vectors so that those PAC and PVC occurrences that are between 2-4 or quadrants 624, 628 are identified and reclassified as PAC and PVC. That is, if the standard deviation of θ₁₂₃ and θ₆₄₅ are less than a first standard deviation value (e.g., 10) and a second standard deviation value (e.g., 10) then the window is flagged as PAC or PVC. The first standard deviation value and the second standard deviation value may be the same value or different values. As an example, a determination of counts n₁₂₃ and n₆₄₅ over a collective reference value and over respective individual reference values, the standard deviation of θ₁₂₃ and θ₆₄₅ is used. Otherwise, the std₆₄₅ and std₁₂₃ is set to zero based on the respective individual reference values. If the standard deviation of θ₁₂₃ and θ₆₄₅ are less than a first standard deviation value (e.g., 10) and a second standard deviation value (e.g., 10) then the window is flagged as PAC or PVC. Otherwise, r_{zero}, the ratio of points found with quadrant 620 with respect to the quantity of points in any other quadrant 622, 624, 626, 628, 630, 632, 634, 636 is used to identify a NSR or an atrial fibrillation according to a atrial fibrillation reference value (e.g., 0.13, 0.5) because NSR rhythms are typically contained within quadrant 620 and atrial fibrillation may be more dispersed.

The collective reference value is used because PAC and PVC often occur more than once and this can improve accuracy as either random motion artifacts or noise can lead to the kite patterns disclosed.

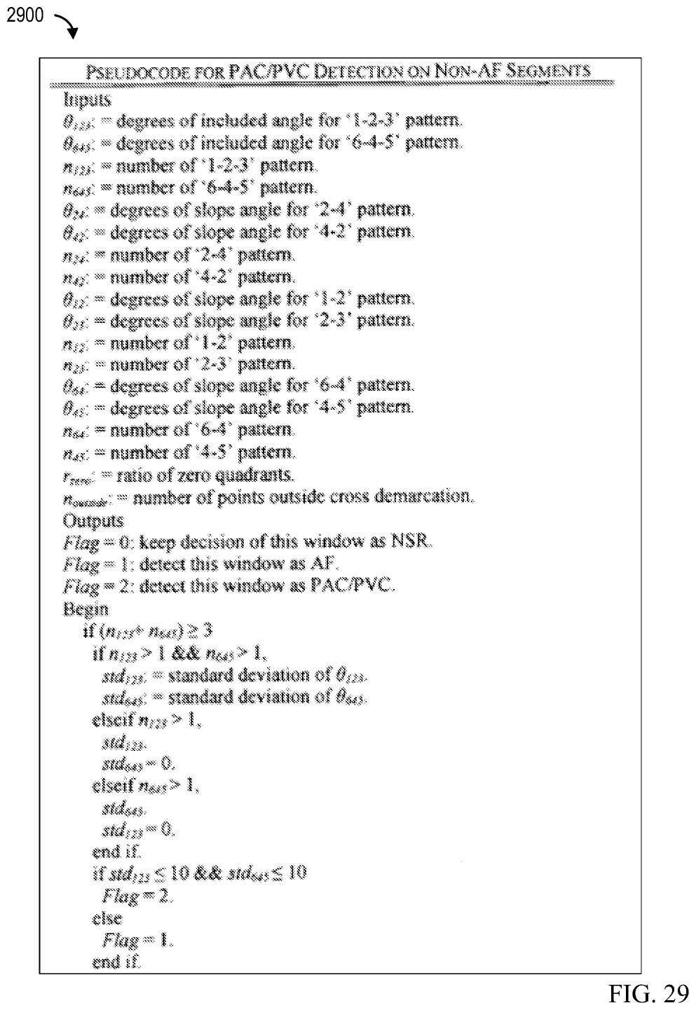

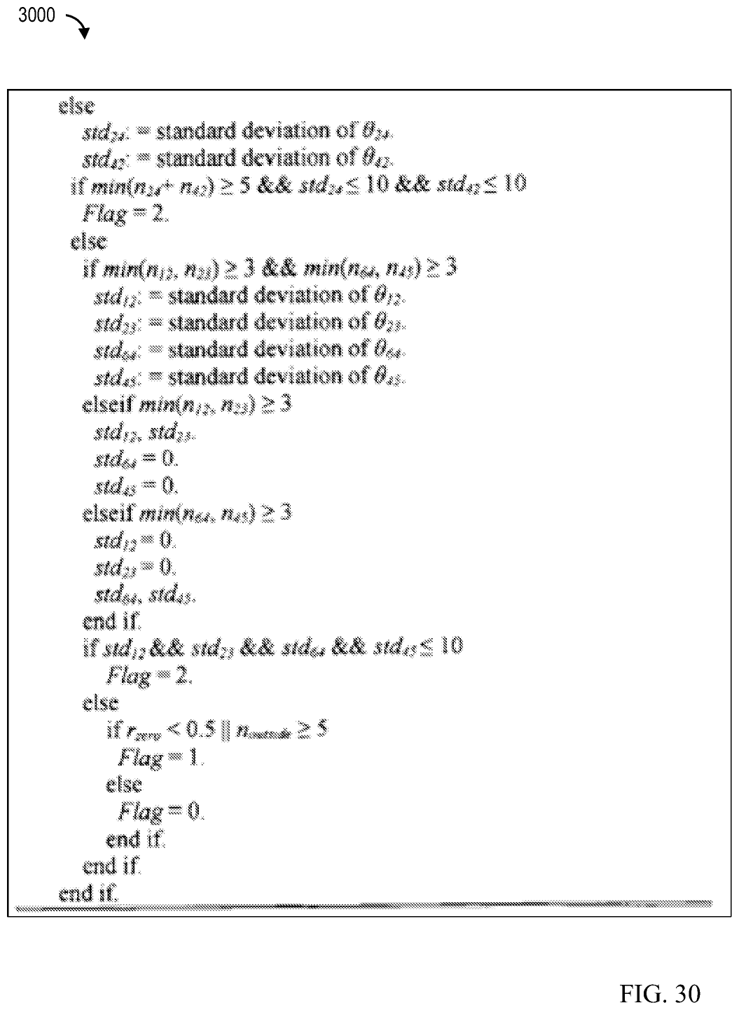

Referring to FIGS. 29-30, pseudocode 2900, 3000 for PAC and PVC detection on non-atrial fibrillation windows 128 is shown in accordance with one or more implementations of the present disclosure. This pseudocode 2900, 3000 has similar inputs to the above references pseudocode 2800. As an example, n₁₂₃, n₆₄₅, θ₁₂₃, θ₆₄₅, n₂₄, n₄₂, θ₂₄, θ₄₂, n₁₂, n₂₃, θ₁₂, θ₂₃, n₆₄, n₄₅, θ₆₄, θ₄₅, r_{zero}, and r_{outside} may be used, where nₓ is the quantity of points without the respective quadrants for the window and θₓ is the angle between the respective vectors or the degree of slope for the respective vector.

Of note, pseudocode 2900, 3000 takes into account 2-4 vectors for windows 128 that are predicted as a normal sinus rhythm related with regard to steps 2404, 224. In a similar way, standard deviation, std₁₂₃, of θ₁₂₃ over the window 128 is calculated along with std₆₄₅, of θ₆₄₅ over the window 128 to sort out similar vectors so that those PAC and PVC occurrences that are between 2-4 or quadrants 624, 628 are identified and reclassified as PAC and PVC. Differently, if both the std₁₂₃ and std₆₄₅ are less than a first standard deviation value (e.g., 10) and a second standard deviation value (e.g., 10), respectively, then the window is flagged as PAC or PVC; otherwise the window 128 is classified as atrial fibrillation, bearing in mind that previous classifications may have designated the window 128 as normal sinus rhythm or non-atrial fibrillation.

For determination between NSR and atrial fibrillation within the pseudocode 2900, 3000, two ratios, r_{zero} and r_{outside} may be used. As an example, r_{zero} may be the ratio of points that fall within quadrant 620 and the entire number of data points. r_{outside} may be the total number of points that fall outside of the quadrants 620, 622, 626, 630, 632, if r_{zero} an r_{outside} may then be compared with respective ratio reference values to determine whether the window 128 is to be classified as NSR or atrial fibrillation.

1-2-3 patterns and 6-4-5 patterns may be interrupted by bigeminy or trigemini beats, which can result in 6-4-2-4-5 quadrant beats or 6-4-0-5 quadrant beats. As such, these sequences may be fragmented into individual vectors (e.g., 1-2, 2-3, 6-4, 4-5) and the standard deviations of the corresponding vector pairs (e.g., 1-2 and 2-3, 6-4 and 4-5) may be used to determine if the window 128 contains PAC or PVC, as indicated by pseudocode 3000. Accordingly, pseudocode 2900, 3000 may provide distinguished results indicative of windows 128 corresponding to atrial fibrillation in step 2218, PAC and PVC in step 2308, and NSR in step 2310.

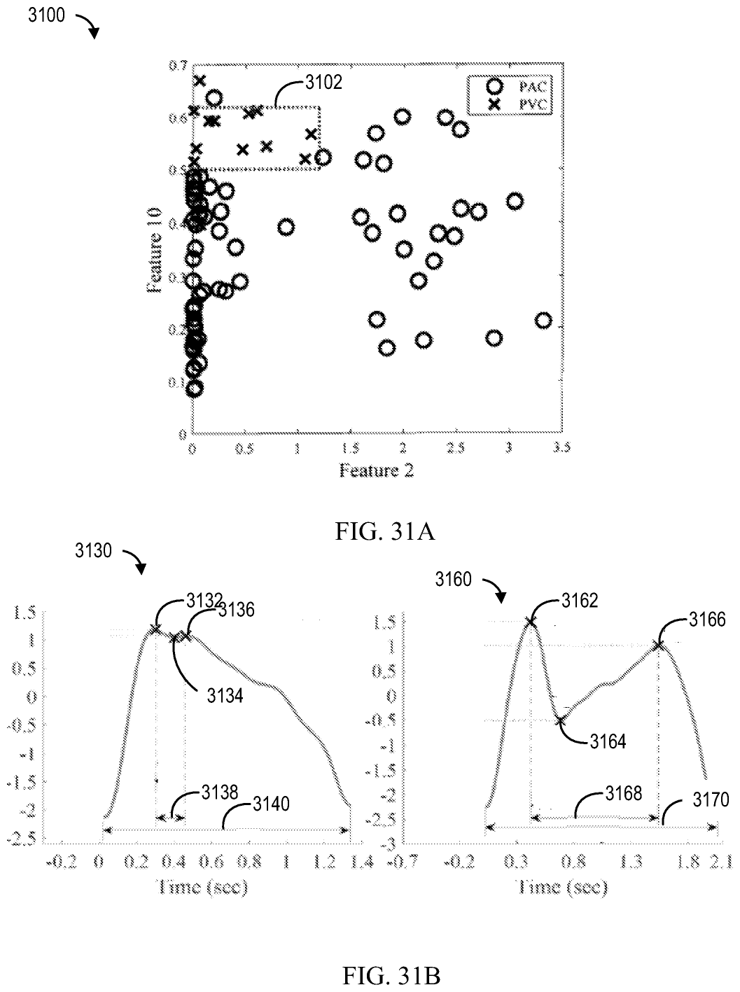

In step 2308 of FIG. 23, an algorithm may be used to distinguish between PAC results in step 2312 and PVC results in step 2314. As an example, FIG. 31A illustrates classification of PAC and PVC in plot 3100 according to features of the underlying PPG signal 126 in accordance with one or more implementations of the present disclosure. As an example, feature 2 is the overall time duration of the PVC beat is statistically longer than PAC beats. In combination with another feature, feature 10, is the prominence of a second peak in the PVC waveform, which occurs after an amplitude of the first peak decreases. Windows 128 having features within PVC box 3102 may be designated as PVC while windows 128 having features outside of the PVC box 3102 may be designated as PAC.

It should be appreciated that normal sinus rhythm (NSR) as discussed herein refers to PPG signals that are not either PAC, PVC, or atrial fibrillation. This application contemplates that NSR may not always be entirely normal or non-indicative of other anomalies not discussed herein.

In FIG. 31B, different PAC and PVC beats are shown in plots 3130, 3160 that demonstrate the features of the underlying PPG signal in accordance with one or more implementations of the present disclosure. As an example, three demarcations 3132, 3134, 3136, 3162, 3164, 3166 may be defined as shown. The prominence of the third demarcations 3136, 3166 may define the feature 2 shown in FIG. 31A indicated by the high difference between respective second demarcations 3134, 3164 and third demarcations 3136, 3166. Feature 10 may be based on the relative time difference between third demarcations 3136, 3166 and first demarcations 3132, 3162, which is the difference between respective first periods 3138, 3168 and second periods 3140, 3170. As such, a determination of PAC and PVC may be based on respective demarcations 3132, 3134, 3136, 3162, 3164, 3166, according to local maxima and local minima determinations and periods 3138, 3168, 3140, 3170 associated therewith. In some cases the non-existence of these local maxima and minima may indicate that the beat and window 128 are PAC.

Although methods of atrial fibrillation, PAC, PVC, and NSR have been discussed, those techniques, may be replaced, improved, or otherwise augmented with feature-based classification for noise detection and denoising techniques. As an example, temporal, morphological, and statistical features may be extracted in time and time-frequency domains. Skewness (Skew), kurtosis (Kurt), pNN40 (percentage of successive beat intervals that differ by more than 40 msec), pNN70, and sample entropy (SampEn) of the PPG signal 126 or windows 128 may be calculated. The RMSSD may be extracted from the intervals between the detected peaks, also known as interbeat intervals (IBI). Further, by transforming the signals from the original time domain to the time-frequency domain, it is possible to observe the variability in the spectral power of the different frequencies over time. In one or more aspects, an applied wavelet transform for each PPG beat after the preprocessing and segmentation procedures to perform time-frequency transformation, in which the approximation and detail coefficients may be obtained. Feature extraction may be obtained by applying wavelet transform on PPG beats: standard deviation (W-STD), skewness (W-Skew), kurtosis (W-Kurt), and average of sub-band energy (W-MSubEn) of the transformed PPG beats.

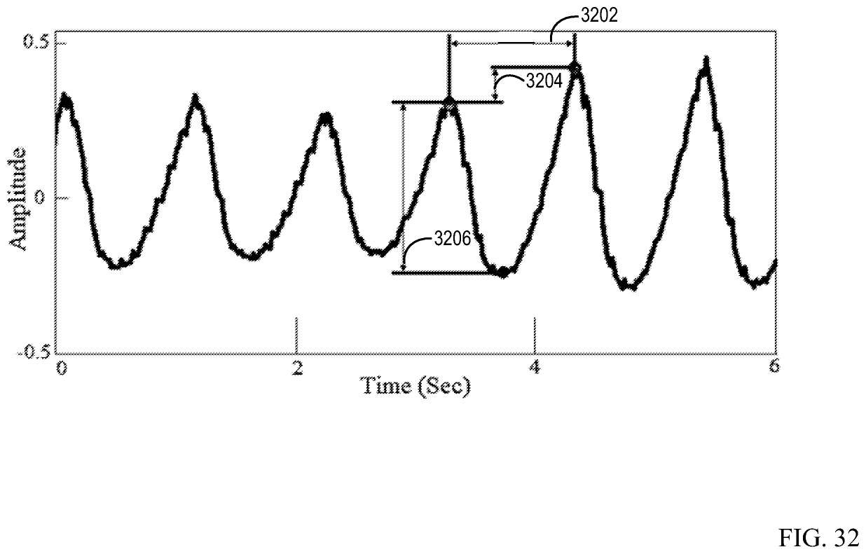

In FIG. 32, three amplitude and time interval measures are defined based on the consecutive PPG signal peaks in accordance with one or more implementations of the present disclosure. As an example, the time interval between two consecutive negative (or positive) peaks 3202 (Tₚ₋ₚ), absolute amplitude difference between two consecutive negative (or positive) peaks 3204 (Aₚ₋ₚ), absolute amplitude difference between negative to positive (or positive to negative) peak 3206 (A). These measures may be calculated for each beat within the window 128 and then, features are extracted from computed measures. As an example, features for each waveform are obtained by calculating the distance of calculated measures from the template segment measures as baseline values. In addition, the beat waveforms between two negative (or positive) peaks may be extracted to measure the dissimilarity of PPG beats from the template beat.

To extract the template-based features from each window 128 or portion thereof, a congruous PPG signal 126 may be necessary over the window 128. Such a window 128 or portion thereof may be used to extract the template baseline values and construct the template beat. An adaptive framework may help to select the template segment, which may also be compatible with the subsequent windows 128 or portions thereof. As an example, a clean PPG segment from the PPG signal 126 may be selected as a template. The PPG segment may be updated contemporaneously to indicate different dynamic characteristics during time. And, the template may be updated for predefined number of segments.

Selecting a clean segment may be achieved through various methods, or combinations thereof, discussed here in. As an example, PPG signal 126 may be scanned for peaks, and number of such peaks may be more than 80% of the length (in sec.) of the window 128 or alternative portion thereof. Amplitudes of the peaks over clean segment may be assumed constant or substantially constant. As such, peak amplitude dispersion may be used as an index to identify the signal quality. Normalized dispersion of the peak amplitudes may be calculated according to Equation 16.

$$D = \frac{s}{μ} \qquad ( 16 )$$

where s and μ are the standard deviation and mean of the peak amplitudes within the window 128, respectively. D, referred to as the Coefficient of Variation (CV) or relative standard deviation (RSD), can indicated the extent of variability in relation to the mean of the peak amplitudes. In a PPG signal, such as PPG signal 126, the CV may be calculated to both the positive and negative peaks according to Equation 17.

D_{comb}=e^{-(|D}^{₁}^{|+|D}^{₂}^{|)} (17)

where D₁ and D₂ are CV values for positive and negative peaks.

The first window 128 of the PPG signal 126 may not be determined as clean. As such, a search to find the first segment may be performed to meet the clean segment criteria. Having the clean segment as the template, the features may be extracted from the previous and subsequent segments, until the next PPG segment satisfies the clean segment criteria to update the template.

Baseline values may be extracted from the uncovered clean template based on Equations 18 and 19.

$$∇ {A}_{p - {p}_{B aseline}} = \frac{{\sum }_{i} {A}_{p - {p}_{{i}_{Temp}} .}}{N - 1} \qquad ( 18 )$$

where Aₚ₋ₚ_{_{i_Temp.}} and N are the absolute value of peak-to-peak amplitude change of beats and the number of peaks in template, respectively, and sum of changes in amplitude is:

$$∇ {A}_{B a s e l i n e} = \frac{{\sum }_{i} {A}_{i - Temp .}}{M - 1} \qquad ( 19 )$$

where A_{i-Temp.} is the absolute value of amplitude change from a negative peak to next positive peak, or a positive peak to the next negative peak, in the template and M is the sum of negative and positive peak numbers in the template.

Using these baselines, features may be extracted as the normalized pulse duration, normalized peak-to-peak amplitude change, and normalized negative to positive, or positive to negative, peak amplitude change for each beat of the PPG signal 126. The normalized pulse duration of a beat, ∇Tₚ₋ₚ_{ᵢ}, may be determined according to Equation 20.

$$∇ {T}_{p - {p}_{i}} = \frac{{T}_{p - {p}_{i}} - ∇ {T}_{p - {p}_{B a s e l i n e}}}{∇ {T}_{p - {p}_{B aseline}}} \qquad ( 20 )$$

where Tₚ₋ₚ_{ᵢ} is the pulse duration of a beat in each window 128 and ∇Tₚ₋ₚ_{_{Baseline}} is the baseline value of pulse duration (peak-to-peak time duration).

To obtain ∇Tₚ₋ₚ_{_{Baseline}}, the heart rate frequency may be determined. The frequency between 1.0 and 3.0 Hz that has the highest power or energy in the spectrum is the heart rate frequency. As an example, ∇Tₚ₋ₚ_{_{Baseline}} may be the inverse of the heart rate frequency, which may be calculated based on the power spectrum of a beat within the template. A normalized peak-to-peak amplitude change (∇Aₚ₋ₚ_{ᵢ}) may be determined according to Equation 21.

$$∇ {A}_{p - {p}_{i}} = \frac{{A}_{p - {p}_{i}} - ∇ A}{∇ A} \qquad ( 21 )$$

where Aₚ₋ₚ_{ᵢ} is the peak-to-peak amplitude change of each beat.

∇A can be considered the normalized negative to positive, or positive to negative, peak change and may be determined according to Equation 22.

$$∇ A = \frac{{\sum }_{i} {A}_{i}}{∇ {A}_{B aseline}} \qquad ( 22 )$$

where Aᵢ and ∇A_{Baseline} are absolute value of amplitude change from a negative peak to next positive peak, or positive peak to next negative peak, for a beat and the template, respectively.

The similarity or dissimilarity may be measured between an averaged beat of the template and each PPG beat as a statistical feature. As an example, dynamic time warping (DTW) may be used to correct the non-stationary time lags between two beats. DTW is a method to calculate the optimal matching between two sequences. All beat waveforms in the PPG segment are normalized into range (e.g., [0 1]) and then, DTW is applied to provide alignment between each normalized beat (B) and average template waveform (T). To extract the similarities, Kullback-Leibler Divergence (KL) divergence is applied to measure the similarity of the aligned waveforms. KL divergence, or relative entropy can quantify how much one probability distribution differs from another distribution.

If P and Q are the n-dimensional probability distribution of discrete random variables, the KL divergence may be determined as shown in Equation 23.

$$KL ( P ‖ Q ) = {\sum }_{i = 1}^{n} {P}_{i} ( x ) log \frac{{P}_{i} ( x )}{{Q}_{i} ( x )} \qquad ( 23 )$$

KL divergence may then be used to extract dissimilarity or a difference between aligned waveforms B and T as a feature represented by D(B,T).

The resulting feature set may contain a total of 134 features, which may be divided into three categories as shown in Table 3. To identify the best feature subset, a comprehensive feature set is desired, and the likelihood of selecting the optimum feature subset when there is a large number of features in the feature set. As such, different types of features may be extracted to constitute the initial feature set.

TABLE 3

| | | Wavelet Domain (cA4, |
|---|---|---|
| Features | Time Domain | cD2, cD3, cD4) |
| Temporal | RMSSD, ∇Tₚ₋ₚ_{ᵢ} | |
| Statistical | Skew, Kurt, pNN4, pNN7, | W-STD, W-Skew, W-Kurt, |
| | SampEn, Dal, n | W-MSubEn |
| Morphological | ∇Aₚ₋ₚ^{ᵢ}, ∇A | |

where cA4, cD2, cD3, and cD4 represent the approximation coefficients scale 4, detail coefficients scale 2, detail coefficients scale 3, and detail coefficients scale 4, respectively.

As the features ∇Tₚ₋ₚ_{ᵢ}, ∇Aₚ₋ₚ_{ᵢ}, ∇A, W-STD, W-Skew, W-Kurt, and W-MSubEn are extracted for each positive and negative PPG beat, their average, standard deviation, skewness, and kurtosis may be calculated for each PPG segment.

Once initial features are extracted from the PPG signals, feature selection may be performed to specify which features are important for noise detection. Two schemes may be used. As an example, filter schemes, the criterion function, may evaluates candidate subsets that are independent of learning algorithms. As another, wrapper schemes may use the performance measure of a classifier model based on the testing results of the candidate subsets to assess the quality of selected features. Embedded methods may use an intrinsic model building measure during learning to embed feature selection into the classification model. A hybrid filter-wrapper feature selection method based on IWSSr algorithm may be used that incorporates a particular classifier.

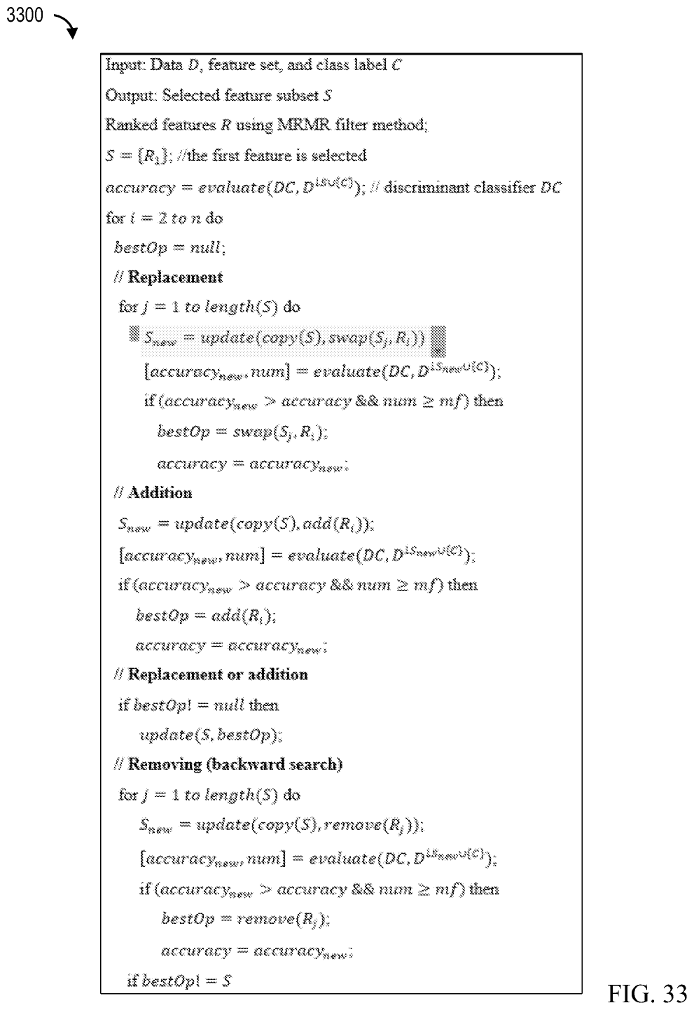

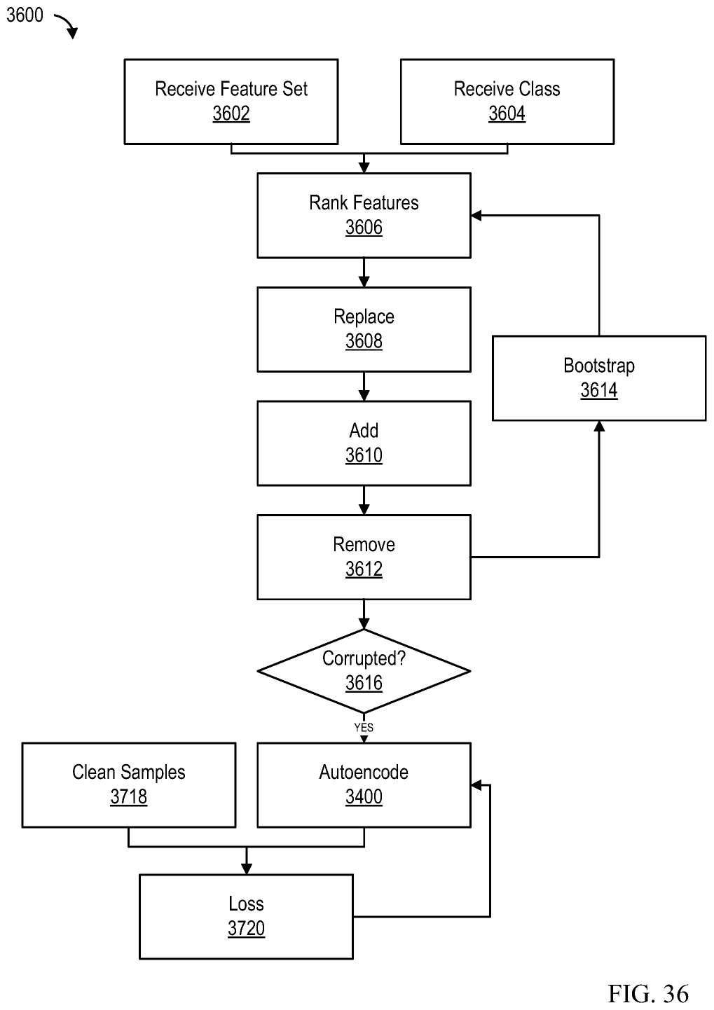

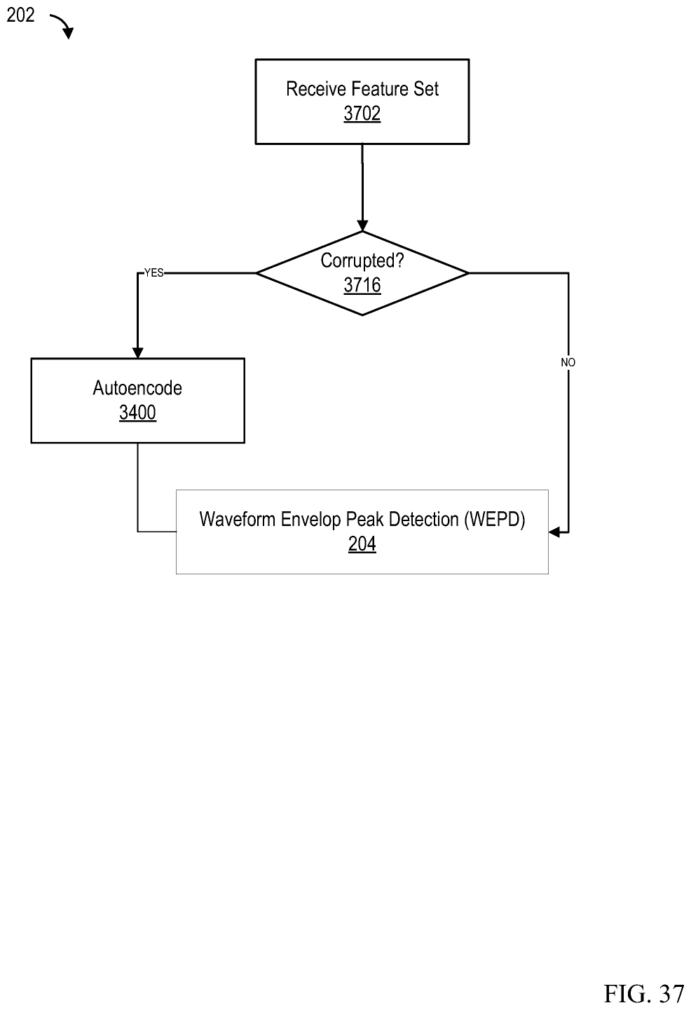

Referring to FIG. 33, a selection method 3300 is shown in accordance with one or more implementations of the present disclosure. The IWSSr selection method uses symmetrical uncertainty (SU) to rank the features based on their relevance to the class labels, which may be received in steps 3602, 3604, 3702 with reference to FIGS. 36-37. An incremental procedure may select an optimal subset of the features. As an example, one feature may be selected at a time from unexplored feature and added to a subset selection less than all of the features based on the performance of the subset selection. A nesting problem may be avoided by checking the next feature selected by the next unexplored feature to determine whether the resultant subset enhances the classification performance. Adding the features to the subset is accomplished repeatedly until no improvement on the subset performance occurs.

An example efficiency improvement may include removing features of the subset selection that do not improve accuracy. As an example, a backward search may be performed to revise the subset selection. As such, as with other techniques disclosed herein, higher computational efficiency is achieved during model training and generalization error is reduced by eliminating the irrelevant features. Another improving technique may include using minimum redundancy and maximum relevance criteria to remove redundant features and rank the features according to relevance before the selection method 3300 commences, which may be performed in step 3606, 3706 with reference to FIGS. 36-37. As an example, the minimum redundancy, maximum relevance (MRMR) method may be used.

As an example, after application of the MRMR method, one relevant feature is added iteratively to the subset selection to create a selected subset of features, which may include replacement of existing features, which may be performed in steps 3608, 3708 with reference to FIGS. 36-37, or addition of new features, which may be performed in steps 3610, 3710 with reference to FIGS. 36-37. In a next phase, a backward search is executed on the subset selection to remove redundant features, which may be performed in steps 3612, 3712 with reference to FIGS. 36-37. The backward search steps may be performed by evaluating the selected features of the subset selection by removing any of the features. The backward search steps may be performed iteratively while performance improves, reducing the size of the subset selection on the iteration and improving the performance of subsequent iterations. As such, a corrupted or clean determination may be performed in steps 3716, 3616 with reference to FIGS. 36-37.

It should be appreciated that bootstrap sampling may be performed, which may be performed in steps 3614 with reference to FIG. 36. Multiple bootstrap subsamples may be created with randomly drawn observations containing a percentage (e.g., 80%) of the training dataset and then, the feature selection method is applied on the subsample. MRMR ranking procedures may be performed on each bootstrap subsample and then, the best features are selected using the selection method 3300. Subsequently, occurrence frequency of each feature may be calculated as the variable importance value. An optimal number of features is specified using the average number of features determined by feature selection method using each bootstrap subsample. Using an optimal feature set, a support vector machine (SVM) classifier or another classifier may be employed to classify the PPG segments into clean and noisy designation.

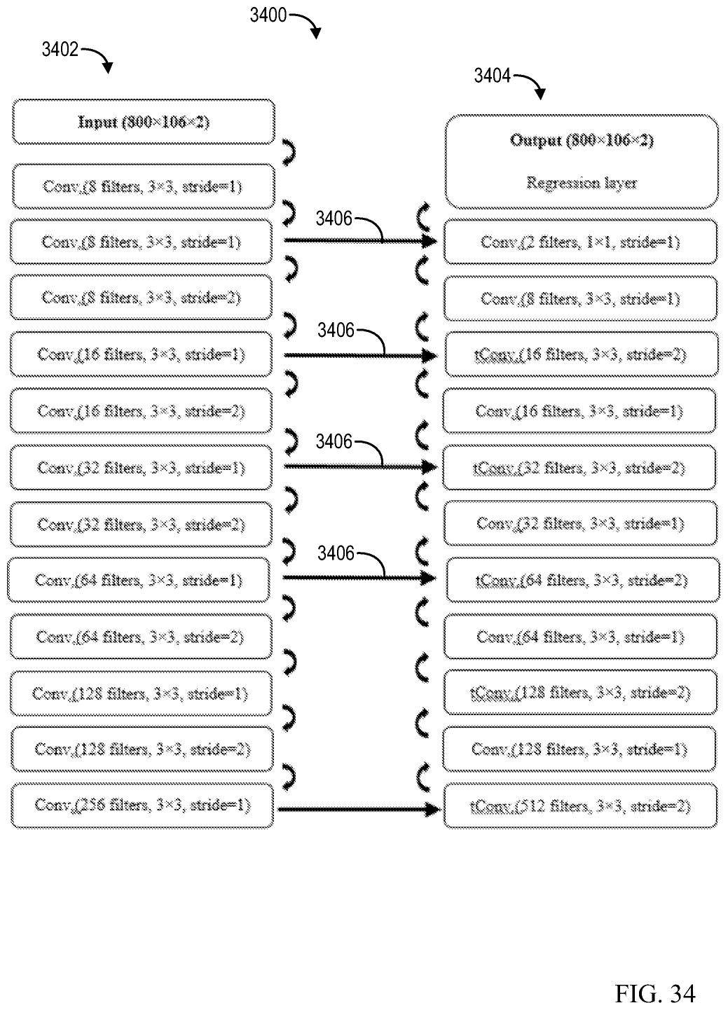

With reference to FIG. 34, an autoencoder 3400 or a network of encoders 3402 and decoders 3404 is shown in accordance with one or more implementations of the present disclosure. As an example, noisy PPG signals 126 or windows 128 may be corrupted, in whole or in part, by noise and motion artifacts. The noisy PPG signals detected by classifier might be fully or partially corrupted by noise. The partially noise-corrupted signals can be recovered using an appropriate denoising approach. An autoencoder 3400 may learn compressed representations of the PPG signal 126 and can be used to recover the noise-corrupted PPG signals 126. The encoders 3402 and decoders 3404 may contain convolutional neural networks (CNN) to remove noise and motion artifacts from the PPG signals 126.

For the encoder, the PPG signal x∈[0,1]^{d} is mapped to a hidden representation y∈[0,1]^{d′} using a deterministic mapping in Equation 24.

y=f_{θ}(x)=s(Wx+b) (24)

where s can be any nonlinear function, W is a d′×d weight matrix, b is a bias vector, and θ={W, b}. Using similar mapping, y is then decoded into a reconstruction z of Equation 25, which is the same shape as x.

z=g_{θ′}(y)=s(W′y+b′) (25)

where W′ is a weight matrix, b′ is a bias vector, and θ′={W′, b′}.

The parameters of this model are optimized to minimize the reconstruction error, which can be evaluated using various loss functions (e.g., squared error, cross-entropy shown in Equation 26).

$${θ}^{*} , {{θ}^{′}}^{*} = {argmin}_{θ , {θ}^{′}} \frac{1}{2} {\sum }_{i = 1}^{n} L ( {x}^{( i )} , {z}^{( i )} ) = {argmin}_{θ , {θ}^{′}} \frac{1}{2} {\sum }_{i = 1}^{n} L ( {x}^{( i )} , {g}_{{θ}^{′}} ( {f}_{θ} ( {x}^{( i )} ) ) ) \qquad ( 26 )$$

The autoencoder 3400 may be trained to reconstruct a signal as noise-free as possible, given the PPG signal 126 as the input and less noisy version as the output. The autoencoder 3400 may have 24 layers and 4 skip connections 3406.

The PPG signal 126 may be transformed into the time-frequency domain using Short-time Fourier transform (STFT) or other methods disclosed herein. Then, the real and imaginary parts of the time-frequency coefficients are obtained and applied as an autoencoder input. The encoder 3402 contains a series of layers, where each individual layer is composed of a convolutional layer, Rectified Linear Unit (ReLU) as activation function, and a batch normalization layer.

The decoder 3404 may be symmetric (in size and operation) to the encoder 3402. Convolutional and transposed convolutional layers may be used in the decoder 3404 to up-sample the feature maps and reconstruct the details. In addition, skip connections 3406 from convolutional layers to their corresponding transposed convolutional layer may be used to enhance the signal recovery performance. Skip connections help to provide the structural details and improve the robustness of the network. The final layer of the model may be a regression model or another model, which generates the output with the real and imaginary parts of the time-frequency coefficients of denoised signal. The denoised PPG signal may be provided by transforming the output into the time domain using the inverse STFT.

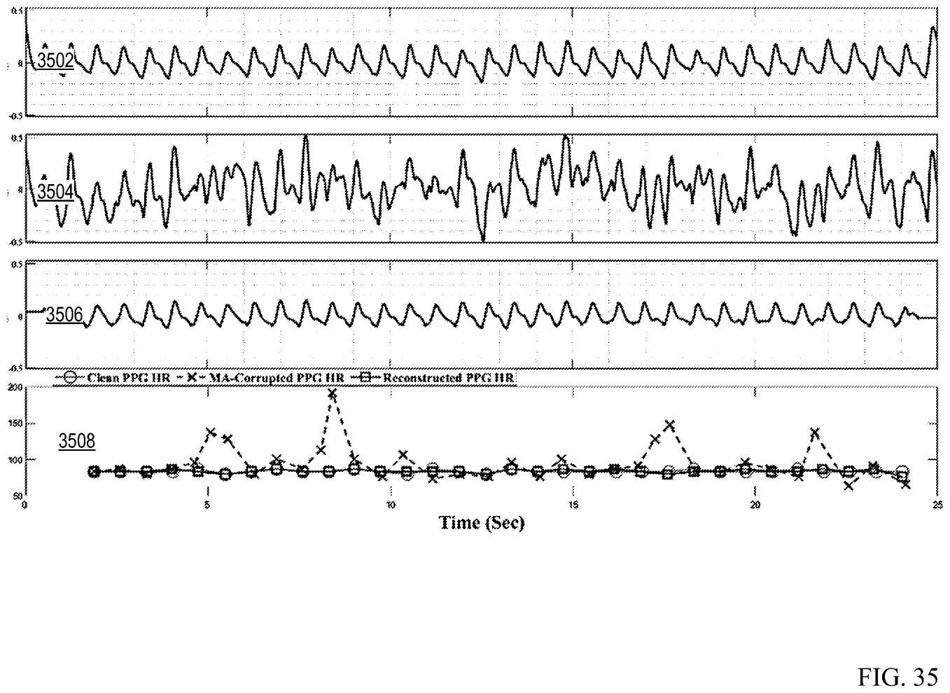

In FIG. 35, a denoised PPG signal 3506 after reconstruction by the autoencoder 3400 is shown in accordance with one or more implementations of the present disclosure. The PPG signal 126 may be received as a clean signal 3502 or a corrupted signal 3504 as shown. As provided in the bottom panel 3508, the denoised PPG signal 3506 resulted in more accurate HR estimates than the HR estimates made using the original PPG signal 126.

In FIG. 36, a training method 3600 is shown in accordance with one or more implementations of the present disclosure. During training, clean samples may be provided in step 3718 of PPG signal 126 to train the autoencoder 3400. A loss calculation may be performed in step 3720 to define weights of the autoencoder 3400. In FIG. 37, a trained model is used to provide the denoised PPG signal 3506 in accordance with one or more implementations of the present disclosure.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
receiving, based on a photoplethysmographic sensor, a photoplethysmographic signal; and
determining an anomaly based on the photoplethysmographic signal relative to a normal sinus rhythm according to a time-frequency representation of the photoplethysmographic signal having a first frequency component, the first frequency component having energies within a first range of frequencies.

2. The method of claim 1, wherein the frequencies are defined according to a heartbeat associated with a heart, the method further comprising:
administering an anti-arrhythmic or defibrillator to treat the heart based on the anomaly.

3. The method of claim 1, wherein the determining the anomaly is according to a first count of the first frequency component according to energy relative a value for the first frequency component.

4. The method of claim 1, wherein the determining the anomaly is according to a first count of the first frequency component according to energy relative a first value for the first frequency component and a second frequency component of the photoplethysmographic signal according to second energy relative a second value for the second frequency component, the second frequency component having second energies within a second range of the frequencies.

5. The method of claim 3, wherein the determining the anomaly is according to a second count of energies having frequencies less than a minimum frequency of a first range or greater than a maximum frequency of the first range, the energies relative a third value.

6. The method of claim 3, wherein the determining the anomaly is according to the first count being greater than a first count reference and the second count being less than a second count reference.

7. The method of claim 1 comprising:
receiving an acceleration signal based on an acceleration sensor, wherein determining the anomaly is according to a characteristic of the acceleration signal relative a value indicative of corruption.

8. The method of claim 1, wherein the determining the anomaly is based on a heartbeat, the heartbeat determined according to steps comprising:
determining that the photoplethysmographic signal is corrupted; and
adjusting the photoplethysmographic signal according to an autoencoder trained to clean corrupted photoplethysmographic signals, the autoencoder comprising an encoder based on a first convolutional network and a decoder based on a second convolutional network.

9. The method of claim 8, wherein the determining that the photoplethysmographic signal is clean comprises:
defining a feature set having features;
classifying the photoplethysmographic signal according to a classifier, the classifier trained according to a subset selection of the features.

10. The method of claim 9, wherein the subset selection is defined by steps comprising:
removing a feature of the subset selection based on an accuracy of the subset selection before the feature is removed relative the accuracy of the subset selection after the feature is removed.

11. The method of claim 10, wherein the subset selection is defined by steps comprising:
adding another feature to the subset selection based on the accuracy of the subset selection before the feature is added relative the accuracy of the subset selection after the feature is added.

12. The method of claim 11, wherein the subset selection is defined by steps comprising:
replacing one or more features of the subset selection based on the accuracy of the subset selection before the feature is replaced relative the accuracy of the subset selection after the feature is replaced.

13. The method of claim 9, wherein the feature set is organized according to a minimum redundancy maximum relevance algorithm.

14. The method of claim 1, wherein the determining the anomaly is based on a heartbeat, the heartbeat determined according to steps comprising:
outputting beat locations based on the photoplethysmographic signal according to local minima of the photoplethysmographic signal.

15. The method of claim 14, further comprising:
removing overlapping beats of the beat locations based on a lowest maximum peak of the beats for a segment of the photoplethysmographic signal.

16. The method of claim 14, further comprising:
removing false positive beats of the beat locations are removed based on an envelope of the beats for a segment of the photoplethysmographic signal.

17. The method of claim 14, further comprising:
inverting a portion of the photoplethysmographic signal over a segment to defined the beat locations.

18. The method of claim 17, wherein the inverted portion of the photoplethysmographic signal is defined according to a quantity of beats in an upper portion of the photoplethysmographic signal and a quantity of beats in a lower portion of the photoplethysmographic signal and the quantity of beats is different by less than ten.

19. The method of claim 14, further comprising:
normalizing the photoplethysmographic signal over a segment to define the beat locations.

20. The method of claim 14, further comprising:
applying a moving average filter to the photoplethysmographic signal over a segment to define the beat locations.

21. The method of claim 1, wherein the determining the anomaly is based on steps comprising:
determining a first difference between a first heart rate defined according to a first beat of the photoplethysmographic signal and a second heart rate defined according to a second beat of the photoplethysmographic signal; and
determining a second difference between the second heart rate and a third heart rate defined according to a third beat of the photoplethysmographic signal.

22. The method of claim 21, wherein a coordinate representation of the first difference and the second difference is characterized according to a quadrant and the anomaly is defined according to the quadrant.

23. The method of claim 1, further comprising:
characterizing beats of the photoplethysmographic signal according to quadrants associated with coordinate representations of first differences defined by the beats and second differences defined by the beats;
defining a first set of angles based on vectors defined by the coordinate representations located within a first set of the quadrants; and
defining a second set of angles based on vectors defined by the coordinate representations located within a second set of the quadrants, wherein determining the anomaly is based on a standard deviation of the first set of angles and a standard deviation of the second set of angles.

24. The method of claim 1, further comprising:
characterizing beats of the photoplethysmographic signal according to quadrants associated with coordinate representations of first differences defined by the beats and second differences defined by the beats;
defining a first quantity based on the coordinate representations located within a first set of the quadrants; and
defining a second quantity based on the coordinate representations located within a second set of the quadrants, wherein determining the anomaly is based on the first quantity and the second quantity.

25. The method of claim 1, further comprising:
characterizing beats of the photoplethysmographic signal according to quadrants associated with coordinate representations of first differences defined by the beats and second differences defined by the beats;
defining a first set of angles based on vectors defined by the coordinate representations located within a first set of the quadrants;
defining a second set of angles based on vectors defined by the coordinate representations located within a second set of the quadrants;
defining a first quantity based on the coordinate representations located within the first set of the quadrants; and
defining a second quantity based on the coordinate representations located within the second set of the quadrants, wherein the anomaly is atrial fibrillation based on the first set, the second set, the first quantity, and the second quantity.

26. The method of claim 1, further comprising:
characterizing beats of the photoplethysmographic signal according to quadrants associated with coordinate representations of first differences defined by the beats and second differences defined by the beats;
defining a first set of angles based on vectors defined by the coordinate representations located within a first set of the quadrants;
defining a second set of angles based on vectors defined by the coordinate representations located within a second set of the quadrants;
defining a first quantity based on the coordinate representations located within the first set of the quadrants; and
defining a second quantity based on the coordinate representations located within the second set of the quadrants, wherein the anomaly is premature ventricular contraction based on the first set, the second set, the first quantity, and the second quantity.

27. The method of claim 1, further comprising:
characterizing beats of the photoplethysmographic signal according to quadrants associated with coordinate representations of first differences defined by the beats and second differences defined by the beats;
defining a first set of angles based on vectors defined by the coordinate representations located within a first set of the quadrants;
defining a second set of angles based on vectors defined by the coordinate representations located within a second set of the quadrants;
defining a first quantity based on the coordinate representations located within the first set of the quadrants; and
defining a second quantity based on the coordinate representations located within the second set of the quadrants, wherein the anomaly is premature atrial contraction based on the first set, the second set, the first quantity, and the second quantity.

28. The method of claim 1, further comprising:
characterizing beats of the photoplethysmographic signal according to quadrants associated with coordinate representations of first differences defined by the beats and second differences defined by the beats;
defining a ratio based on the coordinate representations located within a first set of the quadrants, wherein the anomaly is premature atrial contraction based on the ratio.

29. The method of claim 1, further comprising:
characterizing beats of the photoplethysmographic signal according to quadrants associated with coordinate representations of first differences defined by the beats and second differences defined by the beats;
defining a ratio based on the coordinate representations located within a region, wherein the anomaly determination is based on the ratio.

30. The method of claim 1, wherein the region is defined to separate a rapid ventricle rate anomalies based on the first differences and the second differences, wherein the anomaly is a rapid ventricle rate.

31. The method of claim 1, wherein the region is defined to separate normal sinus rhythms from atrial fibrillations based on the first differences and the second differences, wherein the anomaly is atrial fibrillation.

32. The method of claim 1, wherein determining the anomaly is based on heartbeats and according to steps comprising:
determining local minima for heartbeats that are adjacent and separate by more than a period;
defining a heartrate based on the local minima.

33. An apparatus comprising:
a photoplethysmographic sensor;
an accelerometer; and
a processor, in communication with the photoplethysmographic sensor and the accelerometer, configured to:
receive, from the photoplethysmographic sensor, a photoplethysmographic signal,
receive, from the accelerometer, an acceleration signal, and
determine, based on the photoplethysmographic signal and the acceleration signal, an anomaly in the photoplethysmographic signal relative to a normal sinus rhythm.

34. The instrument of claim 32, wherein the determination of the anomaly is according to a characteristic of the acceleration signal relative a value indicative of corruption and a frequency component of the photoplethysmographic signal.

35. The instrument of claim 32, wherein the determination of the anomaly is according to a time-frequency representation of the photoplethysmographic signal having a first frequency component, the first frequency component having energies within a first range of frequencies defined according to a heartbeat.

36. The instrument of claim 35, wherein the determination of the anomaly is according to a first count of the first frequency component according to energy relative a value for the first frequency component.

37. The instrument of claim 35, wherein the time-frequency representation is a variable frequency complex demodulation of the photoplethysmographic signal.

38. The instrument of claim 36, wherein the determination of the anomaly is according to a second count of energies having frequencies less than a minimum frequency of a first range or greater than a maximum frequency of the first range, the energies relative a third value.

39. The instrument of claim 38, wherein the determination of the anomaly is according to the first count being greater than a first count reference and the second count being less than a second count reference.

40. The instrument of claim 32, wherein the determination of the anomaly is according to a first count of the first frequency component according to energy relative a first value for the first frequency component and a second frequency component of the photoplethysmographic signal according to second energy relative a second value for the second frequency component, the second frequency component having second energies within a second range of the frequencies.

41. The instrument of claim 32, wherein the processor is further operable upon execution of instructions to operate a defibrillator based on the anomaly.

42. The instrument of claim 41, further comprising: the defibrillator operable to treat atrial fibrillation.

43. The instrument of claim 32, wherein the determination of the anomaly is based on a window of the photoplethysmographic signal.

44. The instrument of claim 43, wherein the determination of the anomaly is according to a time-frequency representation of the photoplethysmographic signal having a first frequency component, the first frequency component having energies within a first range of frequencies defined according to a heartbeat and the first frequency component is discontinuous over the window.

45. The instrument of claim 43, wherein the determination of the anomaly is according to a time-frequency representation of the photoplethysmographic signal having a first frequency component, the first frequency component having energies within a first range of frequencies defined according to a heartbeat and the first frequency component is discontinuous over the window.
